(19) United States
(12) Patent Application Publication
(10) Pub. No.: US 2024/0365810 A1
(43) Pub. Date: Nov. 7, 2024

(54) METHOD OF PREPARING A PHOSPHOLIPID-ENRICHED, WHEY-DERIVED COMPOSITION HAVING A LOW CONTENT OF MICROORGANISMS, THE COMPOSITION AS SUCH, AND NUTRITIONAL USE OF THE COMPOSITION

(71) Applicant: ARLA FOODS AMBA, Viby J (DK)

(72) Inventors: Hans BERTELSEN, Viby J (DK); Nikolaj DRACHMANN, Viby J (DK); Anne Staudt KVISTGAARD, Viby J (DK); Kristian Raaby POULSEN, Viby J (DK); Donata VOSYLE, Viby J (DK)

(21) Appl. No.: 18/580,988

(22) PCT Filed: Jul. 18, 2022
(86) PCT No.: PCT/EP2022/070107

§ 371 Date: Jan. 19, 2024

(30) Foreign Application Priority Data
Jul. 20, 2021 (EP) 21186653.8

(51) Int. Cl. A23J 1/20; A23C 21/08; A23L 33/00; A23L 33/12; A23L 33/19

(52) U.S. Cl. CPC A23J 1/205; A23C 21/08; A23L 33/12; A23L 33/19; A23L 33/40; A23C 2210/206

(57) ABSTRACT

The invention relates to a method of preparing a whey-derived composition enriched with respect to whey phospholipids by membrane filtration, and preferably also enriched with respect to other membrane components from whey. The membrane filtration is furthermore followed by a special microfiltration-based germ filtration which the inventors have found to be efficient for reducing the microbial content of the whey-derived composition while keeping nutritionally important components in the whey-derived composition. The invention furthermore relates to the whey-derived composition as such, use of the whey-derived composition as in ingredient in nutritional products, and to nutritional products comprising the whey-derived composition.

### FIELD OF THE INVENTION

The invention relates to a method of preparing a whey-derived composition enriched in whey phospholipids by membrane filtration. The invention furthermore relates to the whey-derived composition as such, use of the whey-derived composition for increasing the content of OPN in nutritional products, and to nutritional products comprising the whey-derived composition.

### BACKGROUND OF THE INVENTION

Milk fat globule membrane (MFGM) is a complex and unique structure composed primarily of lipids and proteins that surrounds milk fat globule secreted from the milk producing cells of humans and other mammals. It is a source of multiple bioactive compounds, including phospholipids, glycolipids, glycoproteins, and carbohydrates that have important functional roles within the brain and gut.

Milk derived milk fat globular membrane components and milk derived functional proteins, in particular phospholipids are known to be valuable additives to nutritious compositions, reported to impact on numerous functions and developments in the human body, including colon cancers, cell growth, brain development, cognitive function and memory.

A method of producing a protein enriched product is known from US2014106044, which discloses a method of producing a protein enriched product, whey protein isolate (WPI), from ultrafiltration of whey to obtain whey protein concentration (WPC) as the retentate and microfiltration of the WPC to produce the protein enriched products as permeate and to recirculate the retentate of the microfiltration.

U.S. Pat. No. 7,259,243 B2 discloses a process for isolation of milk osteopontin from a material containing milk osteopontin by optionally mixing the milk material with a calcium source and separate the osteopontin-containing phase from the rest of the milk material by pH adjustment.

Rocha-Mendoza et al (“Invited review: Acid whey trends and health benefits”, Journal of Dairy Science; vol. 104, no. 2, 23 Dec. 2020, p. 1262-1275) discusses various trends in relation to product, uses and health benefits of acid whey.

US 2019/0388518 A1 disclose formulations having a protein component, in which the protein contains one or more digestion-aiding proteins, and one or more immunoprotective proteins. The ratio by weight of the one or digestion-aiding proteins to the one or more immunoprotective proteins may be about 12:1 to about 1:1. The formulations may also contain a fat component, a carbohydrate component, and vitamins and minerals. These formulations can be used to provide nutritional support to a subject, either as dietary supplements or as a primary source of nutrition, such as for an infant formula. The formulations may also be used to promote or induce proliferation of intestinal cells, promote or induce differentiation of intestinal cells, prevent or inhibit growth of enteropathogenic Escherichia coli in the digestive system of a subject, prevent or inhibit bacterial growth in the intestinal lumen, increase interleukin-18 secretion by intestinal cells, or increase intestinal immunity.

### SUMMARY OF THE INVENTION

The inventors have found that it is challenging to provide high phospholipid whey products with acceptable microbiology without destroying the product. They have found that too harsh heat-treatments seem to denature or degrade some of the bioactive components of the high phospholipid whey products and they have found that traditional germ filtration tends to change the composition of the product too much. However, they discovered that germ filtration using a membrane with a pore size in the range of 1.0-2 micrometers surprisingly could be used for germ filtration which hardly any change in contents of e.g. phospholipids and protein of the whey-derived composition and furthermore found that if this microfiltration is combined with a gentle heat-treatment during sub-step iii) a whey-derived composition with a surprisingly low content of microorganisms is obtained.

Thus, an aspect of the invention pertains to a method of preparing a whey-derived composition enriched with respect to phospholipid and optionally also osteopontin, and preferably also enriched with respect to other milk fat globule membrane components, the method comprising the steps of:
a) providing a liquid feed comprising whey protein including alpha-lactalbumin (ALA) and phospholipid originating from whey, the liquid feed preferably having a pH in the range of 4.0-8, more preferably 5.5-7.5, even more preferably 5.7-7.0, and most preferably 5.9-6.6.
b) subjecting the liquid feed to membrane filtration to provide a filtration retentate and a filtration permeate, said membrane filtration is arranged and operated to:
   provide a content of total phospholipid relative to total solids of the filtration retentate that is at least 200% of the content of total phospholipid relative to total solids of the liquid feed, and
   provide a content of alpha-lactalbumin on total protein basis of the filtration retentate that is at most 75% of the content of alpha-lactalbumin on total protein basis of the liquid feed, and
c) subjecting the filtration retentate or a product stream comprising at least lipid and protein originating from the filtration retentate to microfiltration using a MF membrane with a pore size in the range of 1.0-2 micrometer and recovering the permeate of the microfiltration, which permeate may be subjected to further processing, and said further processing preferably comprising one or more additional processing steps, preferably comprising one or more of:
   ii) concentration,
   iii) heat-treatment, and
   iv) drying.

It is particularly preferred that the method of preparing a whey-derived composition enriched in whey phospholipids and optionally also osteopontin is performed as a continuous method, i.e. in continuous operation. The inventors have found that continuous operation improves the overall energy efficiency of the process and provides a product with reduced microbial contamination relative to e.g. prolonged batch processes. However, batch or semi-batch implementation is also feasible and can provide products of acceptable quality.

In the above-mentioned aspect of the invention it is sometimes preferred but by no means necessary for the present invention that the membrane filtration of step b) also enriches osteopontin.

An alternative aspect of the invention pertains to a method of preparing a whey-derived composition enriched with respect to phospholipid and osteopontin (OPN), and preferably also enriched with respect to other milk fat globule membrane components, the method comprising the steps of:
a) providing a liquid feed comprising whey protein including osteopontin and alpha-lactalbumin (ALA) and phospholipid originating from whey, the liquid feed containing a total amount of osteopontin in the range of 0.2-2.0% w/w relative to total protein,
b) subjecting the liquid feed to membrane filtration to provide a filtration retentate and a filtration permeate, preferably wherein said membrane filtration is arranged and operated to:
   provide a content of osteopontin on total protein basis of the filtration retentate that is at least 150% of the content of osteopontin on total protein basis of the liquid feed, and
   provide a content of alpha-lactalbumin on total protein basis of the filtration retentate that is at most 75% of the content of alpha-lactalbumin on total protein basis of the liquid feed, and
optionally, c) subjecting the filtration retentate or a product stream comprising at least lipid and protein originating from the filtration retentate to one or more additional processing steps, preferably comprising one or more of:
   i) microfiltration,
   ii) concentration,
   iii) heat-treatment, and
   iv) drying.

Another aspect of the invention pertains to a whey-derived composition comprising:
total lipid in the amount of 10 to 30% w/w relative to total solids,
total phospholipid in an amount of 3 to 12% w/w relative to total solids,
an ash content in the range of 1-10% w/w relative to total solids,
lactose in the amount of at most 10% w/w relative to total solids;
total protein in an amount of 65 to 80% w/w relative to total solids, and
a content of colony forming units (CFU) of at most 10000 CFU/g total solids.

An alternative aspect of the invention pertains to a whey-derived composition comprising:
total lipid in the amount of 10 to 30% w/w relative to total solids,
total phospholipid in an amount of 3 to 12% w/w relative to total solids,
an ash content in the range of 1-10% w/w relative to total solids,
lactose in the amount of at most 10% w/w relative to total solids;
total protein in an amount of 65 to 80% w/w relative to total solids, and
OPN in an amount of 0.8-5% w/w relative to total protein.

The whey-derived composition of the invention is suitable as food ingredient. It is preferably used as an ingredient for production of a paediatric product, more preferably an infant formula or alternatively an ingredient for production of nutritional composition e.g. for adult nutrition.

A further aspect of the invention pertains to a nutritional product, which preferably is a paediatric product, and more preferably an infant formula, comprising the whey-derived composition of the invention in an amount sufficient to:
provide OPN in an amount of at least 10 mg/100 g total solids of the nutritional product, more preferably at least 20 mg/100 g total solids, even more preferably at least 30 mg/100 g total solids, and most preferably at least 40 mg/100 g total solids, and/or
provide vitamin B12 in an amount of at least 0.02 microgram/100 g total solids of the nutritional product, more preferably at least 0.05 microgram/100 g total solids, even more preferably at least 0.10 microgram/100 g total solids, and most preferably at least 0.15 microgram/100 g total solids, and/or
provide a content of solids to the nutritional product of at least 0.1 g/100 g total solids of the nutritional product, more preferably at least 0.5 g/100 g total solids, even more preferably at least 2 g/100 g total solids, and most preferably at least 3 g/100 g total solids.

### BRIEF SUMMARY OF THE FIGURE

FIG. 1 shows the standard curve for determining the content of phospholipid derived from extracellular vesicles relative to total phospholipid of a product originating from whey.

### DETAILED DESCRIPTION

An aspect of the invention pertains to a method of preparing a whey-derived composition enriched with respect to phospholipid and optionally also osteopontin, and preferably also enriched with respect to other milk fat globule membrane components, the method comprising the steps of:
a) providing a liquid feed comprising whey protein including alpha-lactalbumin (ALA) and phospholipid originating from whey, the liquid feed preferably having a pH in the range of 4.0-8, more preferably 5.5-7.5, even more preferably 5.7-7.0, and most preferably 5.9-6.6.
b) subjecting the liquid feed to membrane filtration to provide a filtration retentate and a filtration permeate, said membrane filtration is arranged and operated to:
   provide a content of total phospholipid relative to total solids of the filtration retentate that is at least 200% of the content of total phospholipid relative to total solids of the liquid feed, and
   provide a content of alpha-lactalbumin on total protein basis of the filtration retentate that is at most 75% of the content of alpha-lactalbumin on total protein basis of the liquid feed, and
c) subjecting the filtration retentate or a product stream comprising at least lipid and protein originating from the filtration retentate to microfiltration using a MF membrane with a pore size in the range of 1.0-2 micrometer and recovering the permeate of the microfiltration, which permeate may be subjected to further processing, and said further processing preferably comprising one or more additional processing steps, preferably comprising one or more of:
   ii) concentration,
   iii) heat-treatment, and
   iv) drying.

In the context of the present invention the term “whey-derived” means that at least the lipid and protein of a given composition originate from whey. Preferably substantially all the solids of the given “whey-derived” composition originate from whey, except for minerals added during pH adjustments.

A component which “originates from” a composition, e.g. from whey, has been provided by that composition, and has typically been provided by processing of the composition, e.g. by mechanical fractionation such as e.g. centrifugation or filtration or by other modifications of the composition.

In the context of the present invention the term “whey” relates to the liquid composition, which is left when casein has been precipitated and/or removed from milk. Casein may e.g. be removed by microfiltration or large pore ultrafiltration providing a liquid permeate which is free or essentially free of micellar casein but contains the native whey proteins. This liquid permeate is sometimes referred to as “ideal whey”, “serum”, or “milk serum”. Casein precipitation may e.g. also be accomplished by acidification of milk and/or by use of rennet enzyme. Several types of whey exist, such as “sweet whey”, which is the whey product produced by rennet-based precipitation of casein, and “acid whey” or “sour whey”, which is the whey product produced by acid-based precipitation of casein. Acid-based precipitation of casein may e.g. be accomplished by addition of food acids or by means of bacterial cultures.

In the context of the present invention the phrase “whey-derived composition enriched with respect to phospholipid and osteopontin” means that the whey-derived composition has a content of phospholipid on a total solids basis and osteopontin on a total protein basis that is higher than in the liquid feed from which the whey-derived composition was prepared.

In the context of the present invention the phrase “whey-derived composition enriched with respect to phospholipid” means that the whey-derived composition has a content of phospholipid on a total solids basis that is higher than in the liquid feed from which the whey-derived composition was prepared.

In the context of the present invention the phrase “whey-derived milk fat globular membrane components” is meant a structure composed primarily of proteins and lipids of bovine milk origin. It comprises a variety of proteins, glycoproteins, phospholipids, and glycolipids.

In the context of the present invention the term “extracellular vesicles” or “EV” has its ordinary meaning. EV are typically lipid bilayer-delimited particles that are naturally released from almost all types of cell and, unlike a cell, cannot replicate. EV typically carry a cargo comprising proteins, nucleic acids, lipids, and/or metabolites. EV are also found in mammal milk. By the term “milk EV” is meant EV originating from mammalian milk, and in the present context typically from ruminant or bovine milk.

In the context of the present invention, the term “ALA” or “alpha-lactalbumin” pertains to alpha-lactalbumin from mammal species, e.g. in native and/or glycosylated forms and includes the naturally occurring genetic variants. Preferably the ALA is ruminant ALA, and more preferably bovine ALA. Preferably, ALA originates from ruminant milk and more preferably from bovine milk. The present term “ALA” or “alpha-lactalbumin” does not encompass denatured ALA.

In the context of the present invention, the term “BLG” or “beta-lactoglobulin” pertains to BLG from mammal species, e.g. in native and/or glycosylated forms and includes the naturally occurring genetic variants. The present term “BLG” or “beta-lactoglobulin” does not encompass denatured BLG such as e.g. unfolded BLG or aggregated BLG. Preferably the BLG is ruminant BLG, and more preferably bovine BLG. Preferably, BLG originates from from ruminant milk, and more preferably from bovine milk.

In the context of the present invention, the term “caseinomacropeptide” or “CMP” is a peptide released from kappa-casein during the rennet-mediated casein coagulation step (through the action of chymosin) typically during in the cheese making process. CMP is e.g. found in the whey fraction, which is known as sweet whey or cheese whey. CMP is sometimes referred to as caseinoglycomacropeptide (cGMP) or glycomacropeptide (GMP). Preferably, CMP originates from ruminant milk and more preferably from bovine milk.

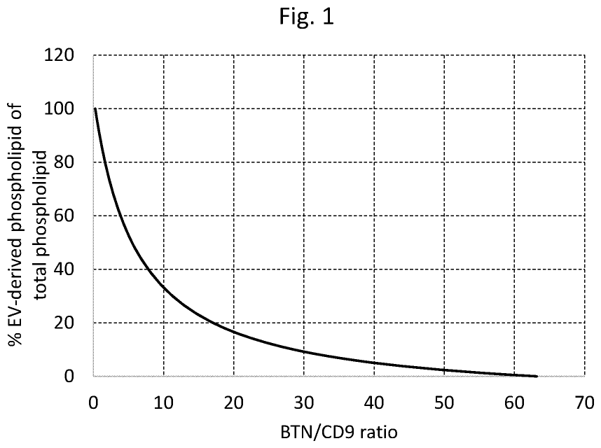

In the context of the present invention, the terms “osteopontin” and “OPN” pertain to both full length osteopontin as found in milk or whey (see e.g. FIG. 1 of FIG. 1 of Christensen et al (B.

Christensen, E. S. Sørensen/International Dairy Journal 57 (2016) 1-6)) including naturally occurring variants and furthermore pertains to long fragments of full length osteopontin which fragments are naturally occurring in milk or whey. The long fragments of OPN that are naturally occurring in milk or whey are typically based on proteolytic cleavage of full length OPN close to the RGD- and SVAYGLR sequence of full length OPN. Preferably the long fragments of OPN are based on cleavage in the region between amino acid position 130 to position 157 of the amino acid sequence of bovine OPN, and more preferably based on cleavage in the boxed region of the amino acid sequence of full length bovine OPN of FIG. 1 of Christensen et al. In the context of the present invention the term “long fragments of OPN” pertain to fragments of full length OPN which fragments contain at least 50 consecutive amino acids from the amino acid sequence of full length OPN, more preferably at least 80 consecutive amino acids, even more preferred at least 90 consecutive amino acids, and most preferably at least 100 consecutive amino acids from the amino acid sequence of full length OPN.

Full-length milk osteopontin is an acidic, highly phosphorylated, sialic acid rich, calcium binding protein. For example, full-length bovine osteopontin contains upto 28 moles of bound phosphate per mol osteopontin and is capable of binding upto approx. 50 moles of Ca per mole osteopontin. OPN is a multifunctional bioactive protein that is implicated in numerous biological processes, such as bone remodelling, inhibition of ectopic calcification, and cellular adhesion and migration, as well as several immune functions. Osteopontin has cytokine-like properties and is a key factor in the initiation of T helper 1 immune responses. Osteopontin is present in most tissues and body fluids, with the highest concentrations being found in milk. Christensen et al is incorporated herein for all purposes. Preferably, the OPN originates from ruminant milk and more preferably from bovine milk.

The content of OPN is quantified according to Analysis 1.

The content of total solids of a composition is quantified according to Example 1.15 of WO 2020/002426.

The content of total protein of a composition is quantified according to Example 1.5 of WO 2020/002426.

The ash content of a composition is quantified according to Example 1.13 of WO 2020/002426 The content of BLG, ALA and CMP are quantified according to Example 1.6 of WO 2020/002426 The pH of a composition is measured according to Example 1.16 of WO 2020/002426.

The content of specific minerals of a composition is quantified according to Example 1.19 of WO 2020/002426.

The content of total lipid of a composition is quantified according to Example 1.27 of WO 2020/002426.

In step a) a liquid feed is provided, which liquid feed comprises whey protein including alpha-lactalbumin (ALA) and phospholipid originating from whey.

In the context of the present invention the term “liquid feed” is used to describe the liquid that is subjected to membrane filtration in step b). Both the protein and the phospholipid of the liquid feed originate from whey, and preferably substantially all solids, of the liquid feed originate from whey.

The whey from which at least the lipid and protein of the liquid feed originate is preferably prepared from ruminant milk and more preferably from bovine milk.

The liquid feed is preferably prepared without drying the lipid and whey protein originating from whey.

The liquid feed preferably has a degree of BLG denaturation of at most 30%, more preferably at most 20%, even more preferably at most 10% and most preferably at most 5%. The degree of BLG denaturation is determined as the percentage of total BLG that is not native BLG. Total BLG and native BLG can be determined by HPLC under reducing conditions for total BLG and under non-reducing conditions for native BLG.

In some preferred embodiments of the present invention the liquid feed comprises or even consists of whey.

The whey is preferably a sweet whey, i.e. obtained from rennet-based casein coagulation, e.g. during cheese production, or an acid whey, i.e. from acid-based casein coagulation, e.g. from the production of caseinate.

The whey is preferably the whey resulting from casein precipitation of whole milk, skimmed milk, or a mixture thereof.

In other preferred embodiments of the present invention the liquid feed comprises or even consists of a protein concentrate of whey.

In the context of the present invention a “protein concentrate” of a whey is a liquid composition in which at least the lipid and protein originate from the whey but which has a higher protein content relative to total solids than the whey. Preferably, substantially all solids of the protein concentrate originate from whey.

It is often preferred that the liquid feed has preferably not been subjected to processing which gives it a reduced content of total phospholipid relative to total solids relative to the whey from which it originates. In this way a higher yield of phospholipids is obtained.

Preferably, the provision of a protein concentrate of whey involves at least partial removal of one or more of:
free carbohydrate,
mineral,
non-protein nitrogen,
particles having a diameter of 1.5 micrometer or larger, and
water,
   from the whey.

In some preferred embodiments of the present invention, the liquid feed is a protein concentrate of whey and the provision of the liquid feed comprises subjecting whey to one or more steps of:
concentration, e.g. by ultrafiltration or nanofiltration
demineralisation, e.g. by diafiltration or electrodialysis
diafiltration, preferably in combination with ultrafiltration or nanofiltration, and
at least partial removal of particles having a diameter of 1.5 micrometer or larger, e.g. by centrifugation or microfiltration using a membrane with a pore size in the range of approx. 1-2 micrometer.

In some preferred embodiments of the invention the liquid feed is prepared by subjecting a whey to:
concentration by ultrafiltration using a membrane having a nominal molecular weight cut-off in the range of 1-10 kDa, optionally in combination with diafiltration, and
at least partial removal of particles having a diameter of 1.5 micrometer or larger, e.g. by centrifugation or microfiltration using a membrane with a pore size in the range of approx. 1-2 micrometer.

In some preferred embodiments of the present invention the liquid feed comprises total protein in an amount in the range of 5-89% w/w relative to total solids, more preferably 30-86% w/w, even more preferably 40-83% w/w, and most preferably 60-80% w/w relative to total solids.

In other preferred embodiments of the present invention, the liquid feed comprises total protein in an amount in the range of 5-25% w/w relative to total solids, more preferably 5-20% w/w, even more preferably 5-15% w/w, and most preferably 5-10% w/w relative to total solids.

Preferably, the liquid feed comprises a total amount of beta-lactoglobulin in the range of 10-70% w/w relative to total protein, more preferably 30-65% w/w, even more preferably 40-60% w/w, and most preferably 45-55% w/w relative to total protein.

Preferably, the liquid feed comprises a total amount of alpha-lactalbumin in the range of 5-40% w/w relative to total protein, more preferably 10-35% w/w, even more preferably 10-30% w/w, and most preferably 10-25% w/w relative to total protein.

In some preferred embodiments of the present invention the liquid feed comprises a total amount of caseinomacropeptide (CMP) in the range of 5-30% w/w relative to total protein, more preferably 10-30% w/w, even more preferably 10-25% w/w, and most preferably 10-20% w/w relative to total protein. The liquid feed typically contains CMP when the whey protein originates from sweet whey.

In other preferred embodiments of the present invention the liquid feed comprises a total amount of caseinomacropeptide (CMP) of at most of 5% w/w relative to total protein, more preferably at most 3% w/w, even more preferably at most % w/w, and most preferably at most 0.5% w/w relative to total protein. The liquid feed typically contains low amounts of CMP or even no CMP when the whey protein originates from e.g. acid whey.

It is sometimes preferred that the liquid feed comprises a total amount of osteopontin in the range of 0.2-2.0% w/w relative to total protein.

In some preferred embodiments of the present invention the liquid feed comprises a total amount of osteopontin in the range of 0.2-0.9% w/w relative to total protein, more preferably 0.3-0.8% w/w, even more preferably 0.4-0.8% w/w, and most preferably 0.4-0.7% w/w relative to total protein. This is often the case when the protein of the liquid feed originates from sweet whey.

In other preferred embodiments of the present invention the liquid feed comprises a total amount of osteopontin in the range of 1.0-2.0% w/w relative to total protein, more preferably 1.2-2.0% w/w, even more preferably 1.3-2.0% w/w, and most preferably 1.4-2.0% w/w relative to total protein. This is often the case when the protein of the liquid feed originates from acid whey.

Preferably, the liquid feed comprises total lipid in an amount in the range of 1-10% w/w relative to total solids, more preferably 2-8% w/w, even more preferably 3-7% w/w, and most preferably 4-7% w/w relative to total solids.

Additionally, the liquid feed preferably comprises a total amount of phospholipid in the range of 10-50% w/w relative to total lipid, more preferably 20-47% w/w, even more preferably 25-45% w/w, and most preferably 29-41% w/w relative to total lipid.

In preferred embodiments of the present invention the liquid feed comprises a total amount of phospholipid in the range of 0.2-5% w/w relative to total solids, more preferably 0.4-4% w/w, even more preferably 0.5-3% w/w, and most preferably 1-3% w/w relative to total solids.

In some preferred embodiments of the present invention the liquid feed comprises a total amount of phospholipid derived from milk extracellular vesicles (milk EV) in an amount of at least 50% w/w relative to total phospholipid, more preferably at least 54% w/w, even more preferably at least 56% w/w, and most preferably at least 58% w/w.

The amount of phospholipid derived from milk EV relative to total phospholipid is determined according to Analysis 2.

It is often preferred that the liquid feed comprises a total amount phospholipid derived from milk EV in an amount of 50-75% w/w relative to total phospholipid, more preferably 54-73% w/w, even more preferably 56-71% w/w, and most preferably 58-70% w/w. The inventors have found these ranges to be typical for liquid feed prepared from sweet whey.

In other preferred embodiments of the invention the liquid feed comprises a total amount phospholipid derived from milk EV in an amount of at least 76% w/w relative to total phospholipid, more preferably at least 80% w/w, even more preferably at least 85% w/w, and most preferably at least 90% w/w. The inventors have found these ranges to be typical for liquid feed prepared from acid whey.

The liquid feed may comprises free carbohydrate, such as e.g. lactose and oligosaccharides as well as carbohydrate bound to e.g. protein or complex lipids such a gangliosides.

In some preferred embodiments of the present invention the liquid feed comprises a total amount of free carbohydrate in the range of 0-85% w/w relative to total solids, more preferably 1-55% w/w, even more preferably 1-50% w/w, and most preferably 1-30% w/w relative to total solids.

The liquid feed preferably comprises a total amount of lactose in the range of 0-80% w/w relative to total solids, more preferably 0-55% w/w, even more preferably 0-50% w/w, and most preferably 0-30% w/w relative to total solids.

Whey is a source of vitamin B12, and preferably, the liquid feed comprises vitamin B12 in an amount in the range of 2-16 microgram/kg total solids, more preferably 4-14 microgram/kg total solids, even more preferably 6-12 microgram/kg total solids, and most preferably 8-10 microgram/kg total solids.

The liquid feed preferably has an ash content in the range of 1-10% w/w relative to total solids, more preferably 1-8% w/w, even more preferably 2-8% w/w, and most preferably 3-7% w/w relative to total solids.

While a broad range of total solids contents may be used in the liquid feed it is preferred that it comprises total solids in an amount of 1-20% w/w relative to the weight of the liquid feed, more preferably 2-15% w/w, even more preferably 4-12% w/w, and most preferably 5-10% w/w relative to the weight of the liquid feed.

The matter of the liquid feed that is not solids is preferably water.

Preferably, the liquid feed comprises total protein in an amount of 0.2-8% w/w relative to the weight of the liquid feed, more preferably 1-7% w/w, even more preferably 2-6% w/w, and most preferably 2-5% w/w relative to the weight of the liquid feed.

The liquid feed preferably has a pH in the range of 4.0-8, more preferably 5.5-7.5, even more preferably 5.7-7.0, and most preferably 5.9-6.6.

In some preferred embodiments of the present invention the liquid feed is a whey protein concentrate (WPC), preferably prepared by at least ultrafiltration.

In the context of the present invention, the terms “whey protein concentrate” (WPC) and “serum protein concentrate” (SPC) pertain to dry or aqueous compositions which contain total protein in an amount of 20-89% w/w relative to total solids.

In the context of the present invention, the term “whey protein” pertains to protein that is found in whey or in milk serum. Whey protein may be a subset of the protein species found in whey or milk serum, and even a single whey protein species or it may be the complete set of protein species found in whey or/and in milk serum.

The term “milk serum protein” or “serum protein” pertains to the protein which is present in the milk serum.

A WPC or an SPC preferably contains total protein in an amount of 20 to 89% w/w relative to total solids, total lipid in an amount of 1 to 10% w/w relative to total solids, an ash content of 1 to 10% w/w of relative to total solids, lactose in an amount of 0 to 70% w/w relative to total solids. Such a WPC or SPC may e.g. comprise 15-70% w/w BLG relative to total protein, 8-50% w/w ALA relative to total protein, and 0-40% w/w CMP relative to total protein.

More preferably a WPC or an SPC may contain total protein in an amount of 35 to 89% w/w relative to total solids, total lipid in an amount of 1 to 10% w/w relative to total solids, an ash content of 2 to 10% w/w of relative to total solids, lactose in the amount of 0 to 60% w/w relative to total solids. Such a WPC or SPC may e.g. comprise 15-70% w/w BLG relative to total protein, 8-50% w/w ALA relative to total protein, and 0-40% w/w CMP relative to total protein.

Even more preferably a WPC or an SPC may contain total protein in an amount of 65 to 89% w/w relative to total solids, total lipid in an amount of 5 to 10% w/w relative to total solids, an ash content of 1 to 5% w/w relative to total solids, lactose in the amount of 0 to 20% w/w relative to total solids. Such a WPC or SPC may e.g. comprise 30-90% w/w BLG relative to total protein, 4-35% w/w ALA relative to total protein, and 0-25% w/w CMP relative to total protein.

SPC typically contain no CMP or only traces of CMP.

Step b) involves subjecting the liquid feed to membrane filtration to provide a filtration retentate and a filtration permeate. The membrane filtration is preferably arranged and operated to:
to provide a content of total phospholipid relative to total solids of the filtration retentate that is at least 200% of the content of total phospholipid relative to total solids of the liquid feed, and
provide a content of alpha-lactalbumin on total protein basis of the filtration retentate that is at most 75% of the content of alpha-lactalbumin on total protein basis of the liquid feed.

The phrase “arranged and operated to” means that the membrane filtration of step b) is implemented and operated with parameters to provide at least the above-mentioned enrichment of phospholipid and the above-mentioned depletion of ALA. General membrane filtration, including its implementation and operation, is well-known to the skilled person and is e.g. described in “Membrane filtration and related molecular separation technologies”, APV Systems, Nielsen W. K. (Ed.), Silkeborg Bogtrykkeri A/S (2003), ISBN 8788016757-9788788016758. Preferred implementations and process parameters have furthermore been described herein as additional guidance to the skilled person.

Step b) may result in multiple filtration permeates or just a single filtration permeate. The filtration permeate(s) of step b) are preferably processed to obtain additional whey protein products.

In some preferred embodiments of the present invention the membrane filtration of step b) is arranged and operated to provide a content of beta-lactoglobulin on total protein basis of the filtration retentate that is at most 100% of the content of beta-lactoglobulin on total protein basis of the liquid feed, more preferably at most 90%, even more preferred at most 80% and most preferred at most 70% of the content of beta-lactoglobulin on total protein basis of the liquid feed.

The smaller whey proteins such as ALA and CMP are removed from the liquid feed at a faster rate than BLG. Therefore in some embodiments, the content of BLG on a total protein basis of filtration retentate is nearly the same as in the liquid feed. This is a result of the more rapid removal of the smaller proteins. The content of OPN on a total protein basis, however, will always increase from the liquid feed to the filtration retentate.

Preferably, the membrane filtration of step b) is arranged and operated to provide a content of beta-lactoglobulin on total protein basis of the filtration retentate that is in the range of 10-100% of the content of beta-lactoglobulin on total protein basis of the liquid feed, more preferably 20-98%, even more preferred 30-96% and most preferred 40-94% of the content of betalactoglobulin on total protein basis of the liquid feed.

Alternatively, but also preferred, the membrane filtration of step b) is arranged and operated to provide a content of beta-lactoglobulin on total protein basis of the filtration retentate that is in the range of 10-90% of the content of beta-lactoglobulin on total protein basis of the liquid feed, more preferably 15-80%, even more preferred 20-70% and most preferred 25-60% of the content of beta-lactoglobulin on total protein basis of the liquid feed.

In some preferred embodiments of the present invention, the membrane filtration of step b) is arranged and operated to provide a content of alpha-lactalbumin on total protein basis of the filtration retentate that is at most 50% of the content of alpha-lactalbumin on total protein basis of the liquid feed, more preferably at most 30%, even more preferred at most 20% and most preferred at most 10% of the content of alpha-lactalbumin on total protein basis of the liquid feed.

Preferably, the membrane filtration of step b) is arranged and operated to provide a content of alpha-lactalbumin on total protein basis of the filtration retentate that is in the range of 5-50% of the content of alpha-lactalbumin on total protein basis of the liquid feed, more preferably 10-45%, even more preferred 15-40% and most preferred 20-35% of the content of alpha-lactalbumin on total protein basis of the liquid feed.

Alternatively, but also preferred, the membrane filtration of step b) may be arranged and operated to provide a content of alpha-lactalbumin on total protein basis of the filtration retentate that is in the range of 1-50% of the content of alpha-lactalbumin on total protein basis of the liquid feed, more preferably 2-30%, even more preferred 3-20% and most preferred 4-10% of the content of alpha-lactalbumin on total protein basis of the liquid feed.

In some preferred embodiments of the present invention, the membrane filtration of step b) is arranged and operated to provide a content of caseinomacropeptide on total protein basis of the filtration retentate that is at most 50% of the content of caseinomacropeptide on total protein basis of the liquid feed, more preferably at most 40%, even more preferred at most 35% and most preferred at most 30% of the content of caseinomacropeptide on total protein basis of the liquid feed.

Preferably, the membrane filtration of step b) is arranged and operated to provide a content of caseinomacropeptide on total protein basis of the filtration retentate that is in the range of 1-50% of the content of caseinomacropeptide on total protein basis of the liquid feed, more preferably 2-40%, even more preferred 3-35% and most preferred 4-30% of the content of caseinomacropeptide on total protein basis of the liquid feed.

Alternatively, but also preferred, the membrane filtration of step b) is arranged and operated to provide a content of caseinomacropeptide on total protein basis of the filtration retentate that is in the range of 1-45% of the content of caseinomacropeptide on total protein basis of the liquid feed, more preferably 2-30%, even more preferred 3-20% and most preferred 4-10% of the content of caseinomacropeptide on total protein basis of the liquid feed.

The inventors have found that, surprisingly, it is technically feasible to provide whey-derived compositions enriched in both whey phospholipids and osteopontin (OPN) by controlled membrane filtration. This was particularly surprising as OPN was expected to be separated from the phospholipids and transferred to the permeate during membrane filtration together with other whey proteins. However, the present inventors found that it is possible to retain OPN in significant amounts in the filtration retentate together with the phospholipids and other MFGM components. This is advantageous as both OPN and the MFGM components are an important nutrients for infant development. The filtration retentate may therefore advantageously be used as an ingredient in infant nutrition as such or converted to a powder ingredient which is better suited for storage and shipping than the liquid filtration retentate.

Thus, it is often preferred that the membrane filtration of step b) is arranged and operated to provide a content of osteopontin on total protein basis of the filtration retentate that is at least 150% of the content of osteopontin on total protein basis of the liquid feed.

In some preferred embodiments of the present invention, the membrane filtration of step b) is arranged and operated to provide a content of osteopontin on total protein basis of the filtration retentate that is at least 180% of the content of osteopontin on total protein basis of the liquid feed, more preferably at least 200%, even more preferably at least 230%, and most preferably at least 250% of the content of osteopontin on total protein basis of the liquid feed.

Preferably, the membrane filtration of step b) is arranged and operated to provide a content of osteopontin on total protein basis of the filtration retentate that is in the range of 150-600% of the content of osteopontin on total protein basis of the liquid feed, more preferably 175-500%, even more preferably 200-450%, and most preferably 225-300% of the content of osteopontin on total protein basis of the liquid feed.

In some preferred embodiments of the present invention, the membrane filtration of step b) is arranged and operated to provide a content of total phospholipid relative to total solids of the filtration retentate that is at least 200% of the content of total phospholipid relative to total solids of the liquid feed, more preferably at least 225%, even more preferably at least 250%, and most preferably at least 275% of the content of total phospholipid relative to total solids of the liquid feed.

Preferably the membrane filtration of step b) is arranged and operated to provide a content of total phospholipid relative to total solids of the filtration retentate that is in the range of 200-600% of the content of total phospholipid relative to total solids of the liquid feed, more preferably 225-550%, even more preferably 250-500%, and most preferably 275-450% of the content of total phospholipid relative to total solids of the liquid feed.

The membrane filtration of step b) typically involves the use of a wide pore ultrafiltration membrane and/or a narrow pore microfiltration membrane.

In some preferred embodiments of the present invention the membrane filtration of step b) involves one or more membrane(s) with a nominal molecular weight cut-off in the range of 100-2000 kDa, more preferably 300-1600 kDa; even more preferably 500-1300 kDa, and most preferably 700-1000 kDa.

The membrane filtration of step b) may furthermore involve the use of one or more additional membranes with lower nominal molecular weight cut-offs than 100 kDa, and such membranes are useful for removing smaller solutes from the liquid feed.

The nominal molecular weight cut-off of a membrane is typically provided by the manufacturer or can be determined according to ASTM standard E 1343-90.

The membrane(s) used in step b) are preferably polymeric membrane. Alternatively, the membrane(s) may be metal membrane(s) or ceramic membrane(s).

More examples on useful membranes may be found in “Membrane filtration and related molecular separation technologies”, APV Systems, Nielsen W. K. (Ed.), Silkeborg Bogtrykkeri A/S (2003), ISBN 8788016757-9788788016758.

Examples of useful membranes are ceramic membranes, organic membranes, polymer membranes, spiral-wound membranes, hollow fibre membranes or flat sheet membranes.

It is presently preferred that the membrane filtration of step b) involves the use of a spiralwound, organic polymer membrane, preferably with a nominal molecular weight cut-off in the range of 500-1300 kDa, and most preferably 700-1000 kDa. A non-limiting example of such a membrane is e.g. FR (PVDF 800 kDa) from Synder Filtration (USA). Membranes with a similar functionality are available from other manufactures.

The membrane filtration of step b) may be implemented in a number of ways and may e.g. involve a single pass filtration or alternatively a series of filtration units, i.e. wherein the liquid feed and any intermediate retentate streams pass multiple membranes arranged in series. The membrane filtration of step b) is typically performed using a filter system arranged for cross flow filtration. Non-limiting examples of useful filter arrangements are spiral-wound filtration systems, hollow fiber membrane systems, and tubular membrane systems.

It is particularly preferred to implement the membrane filtration of step b) as cross flow filtration.

It is furthermore preferred that the membrane filtration of step b) involves diafiltration, preferably wherein one or more intermediate retentate stream(s) are diluted with one or more diluent(s) during step b). The diluent(s) are preferably water and/or protein-free filtration permeates. Such protein-free filtration permeates are preferably prepared by ultrafiltration, nanofiltration, or reverse osmosis. The diafiltration is typically continued until the desired reduction in e.g. ALA has been obtained.

In some preferred embodiments of the present invention, the intermediate retentate stream(s) during step b) comprise total protein in an amount of 0.5-10% w/w relative to the weight of the intermediate retentate stream, more preferably 1-8% w/w, even more preferably 2-7% w/w, and most preferably 2-5% w/w relative to the weight of the intermediate retentate stream.

Preferably, the membrane filtration of step b) is operated with a trans-membrane pressure of 0.1-5 bar, more preferably 0.2-3 bar and most preferably 0.3-1 bar.

Preferably, the membrane filtration of step b) is operated at a temperature of 1-60 degrees C., more preferably 2-30 degrees C., even more preferably 5-20 degrees C., and most preferably 8-15 degrees C.

The temperature of the liquid feed during the step b) may vary within a broad range, but typically it is preferred that the temperature is within the range of 1-60 degrees C. For example, the temperature of the liquid feed during step b) may be in the range of 2-30 degrees C., preferably in the range of 5-20 degrees C., an even more preferred in the range of 8-15 degrees C.

It is presently preferred to keep the temperature of the liquid feed in the lower end of the above-mentioned intervals. Thus, in some preferred embodiments of the invention the temperature of the liquid feed during step b) is in the range of 5-20 degrees C., more preferably in the range of 7-16 degrees C., and most preferred in the range of 8-12.

It is preferred that the temperature of the intermediate retentate stream(s) and the final product stream of the method, except during pasteurisation and spray-drying, are kept within the range of 1-60 degrees C., more preferably 2-30 degrees C., even more preferably 5-20 degrees C., and most preferably 8-15 degrees C.

The filtration retentate preferably comprises:
total lipid in the amount of 10 to 30% w/w relative to total solids,
total phospholipid in an amount of 3 to 12% w/w relative to total solids,
an ash content in range of 1-10% w/w relative to total solids,
lactose in the amount of at most 10% w/w relative to total solids;
total protein in an amount of 65 to 80% w/w relative to total solids,
optionally, OPN in an amount of 0.8-5% w/w relative to total protein.

Preferably, the filtration retentate comprises total protein in an amount in the range of 66-78% w/w relative to total solids, more preferably 68-76% w/w, and most preferably 70-76% w/w relative to total solids.

In some preferred embodiments of the present invention, the filtration retentate comprises a total amount of beta-lactoglobulin in the range of 10-45% w/w relative to total protein, more preferably 15-40% w/w, even more preferably 20-40% w/w, and most preferably 25-35% w/w relative to total protein.

In some preferred embodiments of the present invention, the filtration retentate comprises a total amount of alpha-lactalbumin in the range of 0-10% w/w relative to total protein, more preferably 0.1-8% w/w, even more preferably 0.3-5% w/w, and most preferably 0.5-3% w/w relative to total protein.

Alternatively but also preferred, the filtration retentate comprises a total amount of alpha-lactalbumin in the range of 1-10% w/w relative to total protein, more preferably 1-9% w/w, even more preferably 2-8% w/w, and most preferably 3-7% w/w relative to total protein.

In some preferred embodiments of the present invention the filtration retentate comprises a total amount of caseinomacropeptide in the range of 0-10% w/w relative to total protein, more preferably 1-8% w/w, even more preferably 2-7% w/w, and most preferably 3-7% w/w relative to total protein.

Alternatively, but also preferred, the filtration retentate comprises a total amount of caseinomacropeptide in the range of 0-9% w/w relative to total protein, more preferably 0.1-7% w/w, even more preferably 0.3-5% w/w, and most preferably 0.5-3% w/w relative to total protein.

In some preferred embodiments of the present invention the filtration retentate comprises a total amount of osteopontin in the range of 0.8-5% w/w relative to total protein, more preferably 0.9-5% w/w relative to total protein, more preferably 1.0-4% w/w, even more preferably 1.1-3% w/w, and most preferably 1.1-1.7% w/w relative to total protein.

In other preferred embodiments of the present invention, the filtration retentate comprises a total amount of osteopontin in the range of 2.0-5% w/w relative to total protein, more preferably 2.2-4.5% w/w, even more preferably 2.5-4.0% w/w, and most preferably 3.0-3.7% w/w relative to total protein.

Preferably, the filtration retentate comprises a total lipid in an amount in the range of 10-29% w/w relative to total solids, more preferably 11-27% w/w, even more preferably 13-25% w/w, and most preferably 16-22% w/w relative to total solids.

Preferably, the filtration retentate comprises a total amount of phospholipid in the range of 10-50% w/w relative to total lipid, more preferably 20-47% w/w, even more preferably 25-45% w/w, and most preferably 29-41% w/w relative to total lipid.

In some preferred embodiments of the present invention, the filtration retentate comprises a total amount of phospholipid in the range of 4-12% w/w relative to total solids, more preferably 4-11% w/w, even more preferably 5-11% w/w, and most preferably 6-10% w/w relative to total solids.

The most prominent phospholipids are typically sphingomyelin (SPH), phosphatidylcholine (PC), and phosphatidylethanolamine (PE). In some embodiments, SPH, PC and PE represent up to 90% of total amount of phospholipids. In preferred embodiments of the invention SPH, PC and PE represent from 50% to 90%, more preferably from 60% to 90%, even more preferably from 70% to 90% and most preferably from 80% to 90% of total amount of phospholipids. Each of the three most prominent phospholipids of the filtration retentate are often present in amounts in the range from 1.2% w/w to 2.5% w/w, such as in the range from 1.5% w/w to 2% w/w. The filtration retentate may additional contain other phospholipids, e.g. phosphatidylinositol (PI) and/or phosphatidylserine (PS).

The phospholipid content of the filtration retentate and other compositions can be analyzed with Phosphorous-31 NMR or various chromatographic methods (e.g., HPLC or GC) known in the art. In some preferred embodiments of the present invention, the filtration retentate comprises a total amount of free carbohydrate in the range of 0-8% w/w relative to total solids, more preferably 0-5% w/w, even more preferably 0-1% w/w, and most preferably 0-0.5% w/w relative to total solids.

Preferably, the filtration retentate comprises a total amount of lactose in the range of 0-8% w/w relative to total solids, more preferably 0-5% w/w, even more preferably 0-1% w/w, and most preferably 0-0.5% w/w relative to total solids.

In some preferred embodiments of the present invention, the filtration retentate comprises vitamin B12 in an amount in the range of 20-60 microgram/kg total solids, more preferably 24-50 microgram/kg total solids even more preferably 26-45 microgram/kg total solids, and most preferably 30-40 microgram/kg total solids.

The inventors have found this to be advantageous for e.g. paediatric nutritional and have seen indications that the combination of whey-derived vitamin B12 and whey phospholipids synergistically supports infant cognitive development.

Preferably, the filtration retentate has an ash content in the range of 0.5-5% w/w relative to total solids, more preferably 1.0-3% w/w, even more preferably 1.5-3% w/w, and most preferably 1.6-2% w/w relative to total solids.

In some preferred embodiments of the present invention the filtration retentate comprises total solids in an amount of 1-30% w/w relative to the weight of the filtration retentate, more preferably 2-15% w/w, even more preferably 4-12% w/w, and most preferably 5-10% w/w relative to the weight of the filtration retentate. This is for example useful for whey-derived compositions in the form of liquid products.

The filtration retentate often comprises total protein in an amount of 0.5-10% w/w relative to the weight of the filtration retentate, more preferably 1-8% w/w, even more preferably 2-7% w/w, and most preferably 2-5% w/w relative to the weight of the filtration retentate.

The matter of the filtration retentate that is not solids is preferably water.

The filtration retentate preferably has a pH in the range of 4.0-8, more preferably 5.5-7.5, even more preferably 5.7-7.0, and most preferably 5.9-6.6.

Typically, the filtration retentate comprises at most 10% w/w casein relative to total solids, preferably at most 5% w/w, more preferred at most 1% w/w, and even more preferred at most 0.5% w/w casein relative to the weight of total solids. The filtration retentate may in some embodiments contain no detectable amount of casein.

Additionally, the filtration retentate preferably comprise cholesterol. The amount of cholesterol is preferably in the range from 3 to 20 mg/g relative to total solids, more preferably in the range from 4 to 15 mg/g, and most preferably in the range from 5 to 10 mg/g relative to total solids.

The filtration retentate preferably comprises gangliosides. The most prominent gangliosides of the filtration retentate are typically GD3 and GM3.

In some preferred embodiments of the present invention, the filtration retentate comprises GD3 in an amount in the range from 1800 to 3800 mg/kg relative to total solids, most preferably 2000 to 3500 mg/kg relative to total solids.

In some preferred embodiments of the present invention the filtration retentate comprises GM3 in an amount in the range from 65 to 90 mg/kg relative to total solids, and most preferably in the range from 70 to 85 mg/kg relative to total solids. The total amount of gangliosides of the filtration retentate may be in the range from 1800 to 4000 mg/kg relative to total solids.

The ganglioside content of the filtration retentate can be analyzed with a LC-MS method, GANGLIO-r-LC-TOF.

Preferably, the filtration retentate comprises Immunoglobulin G (IgG; such as e.g. IgG1 and IgG2) in the range from 1% w/w to 10% w/w relative to total solids, and more preferably in the range from 3% w/w to 8% w/w relative to total solids. The amount of IgG can be analyzed with radial immunodiffusion.

In some embodiments, the filtration retentate comprises bovine serum albumin (BSA). BSA is preferably present in an amount of 1-5% relative to total solids, and most preferably 2% w/w to 3.5% w/w relative to total solids.

In some embodiments, the filtration retentate may also comprise glycosylation-dependent cell adhesion molecule (PP3). The PP3 may e.g. be present in the amount from 1% w/w to 3.5% w/w relative to the total solids of the filtration retentate.

In some embodiments, the filtration retentate may also comprise lactotransferrin (or lactoferrin). Lactoferrin may e.g. be present in an amount from 1% w/w to 1.6% w/w relative the total solids of the filtration retentate.

The filtration retentate may further comprise other membrane components.

The method furthermore comprises step c), i.e. subjecting the filtration retentate or a product stream comprising at least lipid and protein originating from the filtration retentate, to one or more additional processing steps comprising i) microfiltration, and preferably furthermore comprising one or more of the sub-steps:
ii) concentration,
iii) heat-treatment, and
iv) drying.

Preferably step c) comprises microfiltering the filtration retentate or a product stream comprising at least lipid and protein originating from the filtration retentate using a MF membrane with a pore size in the range of 1.0-2 micrometer, and most preferably in the range of 1.2-1.8 micrometer.

The microfiltration of sub-step i) has the purpose of reducing the content of microorganisms and further more reduces residual milk fat globules and large aggregates that were not removed earlier during the method. The permeate of the microfiltration of sub-step i) is recovered and may be subjected to further processing, preferably using one or more of the abovementioned sub-steps.

In the context of the present invention the phrase “product stream comprising at least lipid and protein originating from the filtration retentate” means that the liquid that is processed in substeps i-iv) may be a slightly modified filtration retentate due to the further processing of step c) but wherein the lipid and protein of the product stream(s) still originate from the filtration retentate.

The slight modification(s) can e.g. be:
partial removal of water and small solutes during the concentration of sub-step ii),
at least partial removal of microrganisms and residual milk fat globules during the microfiltration of sub-step i),
partial denaturation during the heat-treatment of sub-step iii), or
addition of water and mineral due to pH adjustments and/or dilutions.

It is furthermore preferred that substantially all solids of the product stream(s) originate from the filtration retentate. Preferably, a product stream during step c) contains at least 90% w/w solids from the filtration retentate relative to the weight of the solids of the product stream, more preferably at least 95% w/w, even more preferably at least 97% w/w, and most preferably at least 99% w/w solids from the filtration retentate relative to the weight of the solids of the product stream.

As mentioned above, the inventors have found that it is challenging to provide high phospholipid whey products with acceptable microbiology without destroying the product. They have found that too harsh heat-treatments seem to denature or degrade some of the bioactive components of the high phospholipid whey products and they have found that traditional germ filtration tends to change the composition of the product too much. However, they discovered that germ filtration using a membrane with a pore size in the range of 1.0-2 micrometers surprisingly could be used for germ filtration which hardly any change in contents of e.g. phospholipids and protein of the whey-derived composition and furthermore found that if this microfiltration is combined with a gentle heat-treatment during sub-step iii) a whey-derived composition with a surprisingly low content of microorganisms is obtained.

The gentle heat-treatment preferably involves heating the liquid to be heat-treated to a temperature of at least 60 degrees C. for a duration sufficient for obtaining at least partial microbial reduction, but wherein the heat-treatment denatures at most 5% of the BLG of the liquid to be heat-treated, more preferably at most 2% of the BLG, even more preferably at most 0.5% of the BLG and most preferably at most 0.1% of the BLG.

In preferred embodiments of the invention, the gentle heat-treatment involves heating the liquid to be heat-treated to a temperature in the range of 62-70 degrees C. with a holding time of 5-180 seconds, or more preferably 62-69 degrees C. with a holding time of 10-180 seconds, and most preferably 62-69 degrees C. with a holding time of 10-120 seconds.

The microfiltration of sub-step i) is preferably performed with diafiltration to wash out as much phospholipid as possible to the microfiltration permeate. Preferably using water as diluent.

Preferably, the microfiltration of step c)-i is operated with a trans-membrane pressure of 0.1-10 bar, more preferably 0.2-5 bar and most preferably 0.3-1 bar.

Preferably, the microfiltration of step c)-i is operated at a temperature of 1-60 degrees C., more preferably 2-30 degrees C., even more preferably 5-20 degrees C., and most preferably 8-15 degrees C.

In some preferred embodiments of the present invention step c) furthermore comprises ii) concentration, preferably using one or more of ultrafiltration, nanofiltration, reverse osmosis, and evaporation.

Concentration by ultrafiltration, nanofiltration, reverse osmosis or a combination thereof is particularly preferred.

Preferably, the concentration of step c)-ii is operated at a temperature of 1-60 degrees C., more preferably 2-30 degrees C., even more preferably 5-20 degrees C., and most preferably 8-15 degrees C.

The liquid to be concentrated is preferably concentrated to a total solids content in the range of 5-30% w/w, more preferably 10-28% w/w, even more preferably 12-26% w/w, and more preferably 14-24% w/w.

In some preferred embodiments of the present invention step c) furthermore comprises iii) heat-treated, preferably comprising heat-treatment to a temperature of at least 60 degrees C. for a duration sufficient for obtaining at least partial microbial reduction.

As mentioned above, it is often preferred to employ a gentle heat-treatment to avoid damaging the product.

The gentle heat-treatment preferably involves heating the liquid to be heat-treated to a temperature of at least 60 degrees C. for a duration sufficient for obtaining at least partial microbial reduction, but wherein the heat-treatment denatures at most 5% of the BLG of the liquid to be heat-treated, more preferably at most 2% of the BLG, even more preferably at most 0.5% of the BLG and most preferably at most 0.1% of the BLG.

In preferred embodiments of the invention, the gentle heat-treatment involves heating the liquid to be heat-treated to a temperature in the range of 62-70 degrees C. with a holding time of 5-180 seconds, or more preferably 62-69 degrees C. with a holding time of 10-180 seconds, and most preferably 62-69 degrees C. with a holding time of 10-120 seconds.

Alternatively but also preferably, heat-treatment may involve heating the liquid to be heat-treated to a temperature of at least 70 degrees C. for a duration sufficient for obtaining at least partial microbial reduction, but wherein the heat-treatment denatures at most 20% of the BLG of the liquid to be heat-treated, more preferably at most 10% of the BLG, even more preferably at most 5% of the BLG and most preferably at most 1% of the BLG.

In preferred embodiments of the invention, the heat-treatment involves heating the liquid to be heat-treated to a temperature in the range of 70-80 degrees C. with a holding time of 1-60 seconds, more preferably 70-76 degrees C. with a holding time of 2-50 seconds, and most preferably 70-74 degrees C. with a holding time of 5-30 seconds.

In some preferred embodiments of the present invention step c) furthermore comprises iv) drying.

The drying of step c)-iv may comprising or even consisting of spray drying, freeze drying, fluid bed drying, drum/roller drying, shelf dryers and/or supercritical drying.

Drying by spray-drying is particularly preferred.

The drying of step c)-iv is preferably performed after any of steps i-iii).

Step c) may furthermore comprise a sub-step of packaging the dried whey-derived composition obtained from sub-step iv).

In some preferred embodiments of the present invention the method furthermore comprises step c), and step c) comprises subjecting the filtration retentate to:
i) microfiltration, preferably using a MF membrane with a pore size in the range of 1.0-2 micrometer, followed by
ii) concentration, preferably using ultrafiltration, nanofiltration and/or reverse osmosis, followed by
iii) heat-treatment, preferably involving heating the liquid to be heat-treated to a temperature of at least 60 degrees C. for a duration sufficient for obtaining at least partial microbial reduction, but wherein the heat-treatment denatures at most 5% of the BLG of the liquid to be heat-treated, and followed by
iv) drying, preferably comprising or even consisting of spray-drying.

It is preferred that the present method does not involve solvent extraction or fluid extraction, such as e.g. supercritical or near critical fluid extraction.

In some preferred embodiments of the present invention, the method comprises the steps of:
a) providing a liquid feed comprising whey protein including alpha-lactalbumin (ALA) and phospholipid originating from whey, the liquid feed preferably having a pH in the range of 4.0-8, more preferably 5.5-7.5, even more preferably 5.7-7.0, and most preferably 5.9-6.6.
b) subjecting the liquid feed to membrane filtration to provide a filtration retentate and a filtration permeate, said membrane filtration is arranged and operated to:
   provide a content of total phospholipid relative to total solids of the filtration retentate that is at least 200% of the content of total phospholipid relative to total solids of the liquid feed, and
   provide a content of alpha-lactalbumin on total protein basis of the filtration retentate that is at most 75% of the content of alpha-lactalbumin on total protein basis of the liquid feed, and wherein the membrane filtration of step b) furthermore:
   involves one or more membrane(s) with a nominal molecular weight cut-off in the range of 100-2000 kDa, more preferably 300-1600 kDa; even more preferably 500-1300 kDa, and most preferably 700-1000 kDa,
   involves diafiltration,
   is operated with a trans-membrane pressure of 0.1-5 bar, more preferably 0.2-3 bar and most preferably 0.3-1 bar,
   is operated at a temperature of 1-60 degrees C., more preferably 2-30 degrees C., even more preferably 5-20 degrees C., and most preferably 8-15 degrees C.,
c) subjecting the filtration retentate or a product stream comprising at least lipid and protein originating from the filtration retentate to microfiltration using a MF membrane with a pore size in the range of 1.0-2 micrometer and recovering the permeate of the microfiltration, which permeate may be subjected to further processing, and said further processing preferably comprising one or more additional processing steps, preferably comprising one or more of:
   ii) concentration,
   iii) heat-treatment, and
   iv) drying.

Another aspect of the invention pertains to a whey-derived composition comprising:
total lipid in the amount of 10 to 30% w/w relative to total solids,
total phospholipid in an amount of 3 to 12% w/w relative to total solids,
an ash content in the range of 1-10% w/w relative to total solids,
lactose in the amount of at most 10% w/w relative to total solids,
total protein in an amount of 65 to 80% w/w relative to total solids;
optionally, OPN in an amount of 0.8-5% w/w relative to total protein, and
a content of colony forming units (CFU) of at most 10000 CFU/g total solids.

Preferably, the whey-derived composition comprises total protein in an amount in the range of 66-78% w/w relative to total solids, more preferably 68-76% w/w, and most preferably 70-76% w/w relative to total solids.

In some preferred embodiments of the present invention, the whey-derived composition comprises a total amount of beta-lactoglobulin in the range of 10-45% w/w relative to total protein, more preferably 15-40% w/w, even more preferably 20-40% w/w, and most preferably 25-35% w/w relative to total protein.

In some preferred embodiments of the present invention, the whey-derived composition comprises a total amount of alpha-lactalbumin in the range of 0-10% w/w relative to total protein, more preferably 0.1-8% w/w, even more preferably 0.3-5% w/w, and most preferably 0.5-3% w/w relative to total protein.

Alternatively but also preferred, the whey-derived composition comprises a total amount of alpha-lactalbumin in the range of 1-10% w/w relative to total protein, more preferably 1-9% w/w, even more preferably 2-8% w/w, and most preferably 3-7% w/w relative to total protein.

In some preferred embodiments of the present invention the whey-derived composition comprises a total amount of caseinomacropeptide in the range of 0-10% w/w relative to total protein, more preferably 1-8% w/w, even more preferably 2-7% w/w, and most preferably 3-7% w/w relative to total protein.

Alternatively, but also preferred, the whey-derived composition comprises a total amount of caseinomacropeptide in the range of 0-9% w/w relative to total protein, more preferably 0.1-7% w/w, even more preferably 0.3-5% w/w, and most preferably 0.5-3% w/w relative to total protein.

In some preferred embodiments of the present invention the whey-derived composition comprises a total amount of osteopontin in the range of 0.9-5% w/w relative to total protein, more preferably 1.0-4% w/w, even more preferably 1.1-3% w/w, and most preferably 1.1-1.7% w/w relative to total protein.

In other preferred embodiments of the present invention, the whey-derived composition comprises a total amount of osteopontin in the range of 2.0-5% w/w relative to total protein, more preferably 2.2-4.5% w/w, even more preferably 2.5-4.0% w/w, and most preferably 3.0-3.7% w/w relative to total protein.

Preferably, the whey-derived composition comprises a total lipid in an amount in the range of 10-29% w/w relative to total solids, more preferably 11-27% w/w, even more preferably 13-25% w/w, and most preferably 16-22% w/w relative to total solids.

Preferably, the whey-derived composition comprises a total amount of phospholipid in the range of 10-50% w/w relative to total lipid, more preferably 20-47% w/w, even more preferably 25-45% w/w, and most preferably 29-41% w/w relative to total lipid.

In some preferred embodiments of the present invention, the whey-derived composition comprises a total amount of phospholipid in the range of 4-12% w/w relative to total solids, more preferably 4-11% w/w, even more preferably 5-11% w/w, and most preferably 6-10% w/w relative to total solids.

As mentioned Example 4, the inventors have discovered that milk extracellular vesicles (milk EV) may be present in significant amounts in the whey-derived composition of the invention (both prior to the drying step and in reconstituted whey-derived powders obtained by spray-drying). Without being bound by theory it is believed that the relatively gentle treatment of the liquid feed and the subsequent product streams, and particularly the combination of germ filtration and gentle heat-treatment, results in a high recovery of intact milk EV while the content of microorganisms very low in the final products. Milk EV are believed to be important for e.g. infant development and are e.g. an abundant source of valuable phospholipids and microRNAs. Scientific literature points to milk miRNAs being central regulators of infant gastrointestinal health and immune system development and it has been shown in the prior art that human EV derived miRNAs survive gastrointestinal passage for recipient cell uptake in the gastrointestinal tract (see more details in Example 5).

Therefore, it is often preferred that whey-derived composition comprises milk EV, and preferably intact, milk EV.

The inventors have found that the milk EV often make a substantial contribution to the total content of phospholipid of the whey-derived compositions of the invention.

In some preferred embodiments of the present invention the whey-derived composition comprises a total amount of phospholipid derived from milk EV in an amount of at least 50% w/w relative to total phospholipid, more preferably at least 54% w/w, even more preferably at least 56% w/w, and most preferably at least 58% w/w.

The amount of phospholipid derived from milk EV relative to total phospholipid is determined according to Analysis 2.

It is often preferred that the whey-derived composition comprises a total amount phospholipid derived from milk EV in an amount of 50-75% w/w relative to total phospholipid, more preferably 54-73% w/w, even more preferably 56-71% w/w, and most preferably 58-70% w/w. The inventors have found these ranges to be typical for whey-derived compositions prepared from sweet whey.

In other preferred embodiments of the invention the whey-derived composition comprises a total amount phospholipid derived from milk EV in an amount of at least 76% w/w relative to total phospholipid, more preferably at least 80% w/w, even more preferably at least 85% w/w, and most preferably at least 90% w/w. The inventors have found these ranges to be typical for whey-derived compositions prepared from acid whey.

As described in Example 5, the inventors have discovered that the present whey-derived composition may contains intact milk microRNA (miRNA). More specifically. it was found that both the whey-derived powder and the whey-derived liquid (prior to spray-drying) of Example 1 contained significant amounts of miRNAs of which 5-6 of the 20 most abundant miRNA species were identical to miRNAs from human milk EVs. Milk-derived miRNAs are known to play important roles in relation to infant development and the present invention therefore enables the preparation of paediatric nutrition, and particularly the preparation of infant nutrition, enriched with respect to miRNA species found in human milk. As conventional infant formulas of today are largely devoid of miRNAs, this represents a ‘humanization gap’. The fact that the present inventors have documented preserved EV structures and miRNAs in both the whey-derived liquids and powders of the invention, emphasizes the suitability of employing the whey-derived powder and the whey-derived liquid for further humanization of infant nutrition in terms of bioactive milk EVs with intact miRNA content.

Therefore, in some preferred embodiments of the invention, the whey-derived composition comprises microRNA (miRNA), more preferably miRNA present in mammal milk, and most preferably miRNA present in bovine milk and/or in human milk.

In some preferred embodiments of the invention, the miRNA comprises a plurality of miRNA species, said plurality of miRNA species comprising at least one miRNA species selected from the group consisting of let-7a-5p, let-7b, let-7f, let-7i, miR-103, miR-16b, miR-191, miR-199a3p, miR-21-5p, miR-223, miR-26a, miR-26b, miR-423-3p, and miR-486.

Preferably, the miRNA comprises a plurality of miRNA species, said plurality of miRNA species comprising let-7a-5p, let-7b, let-7f, let-7i, miR-103, miR-16b, miR-191, miR-199a-3p, miR-21-5p, miR-223, miR-26a, miR-26b, miR-423-3p, and miR-486.

In other preferred embodiments of the invention, the miRNA comprises a plurality of miRNA species, said plurality of miRNA species comprising at least one miRNA species selected from the group consisting of let-7a-5p, let-7b, let-7f, miR-191, miR-21-5p, and miR-26a.

Preferably, the miRNA comprises a plurality of miRNA species, said plurality of miRNA species comprising the miRNA species let-7a-5p, let-7b, let-7f, miR-191, miR-21-5p, and miR-26a.

The most prominent phospholipids are typically sphingomyelin (SPH), phosphatidylcholine (PC), and phosphatidylethanolamine (PE). In some embodiments, SPH, PC and PE represent up to 90% of total amount of phospholipids. In preferred embodiments of the invention SPH, PC and PE represent from 50% to 90%, more preferably from 60% to 90%, even more preferably from 70% to 90% and most preferably from 80% to 90% of total amount of phospholipids. Each of the three most prominent phospholipids of the whey-derived composition are often present in amounts in the range from 1.2% w/w to 2.5% w/w, such as in the range from 1.5% w/w to 2% w/w. The whey-derived composition may additional contain other phospholipids, e.g. phosphatidylinositol (PI) and/or phosphatidylserine (PS).

The phospholipid content of the whey-derived composition can be analyzed with Phosphorous-31 NMR or various chromatographic methods (e.g., HPLC or GC) known in the art.

In some preferred embodiments of the present invention, the whey-derived composition comprises a total amount of free carbohydrate in the range of 0-8% w/w relative to total solids, more preferably 0-5% w/w, even more preferably 0-1% w/w, and most preferably 0-0.5% w/w relative to total solids.

Preferably, the whey-derived composition comprises a total amount of lactose in the range of 0-8% w/w relative to total solids, more preferably 0-5% w/w, even more preferably 0-1% w/w, and most preferably 0-0.5% w/w relative to total solids.

In some preferred embodiments of the present invention, the whey-derived composition comprises vitamin B12 in an amount in the range of 20-60 microgram/kg total solids, more preferably 24-50 microgram/kg total solids even more preferably 26-45 microgram/kg total solids, and most preferably 30-40 microgram/kg total solids.

The inventors have found this to be advantageous for e.g. paediatric nutritional and have seen indications that the combination of whey-derived vitamin B12 and whey phospholipids synergistically supports infant cognitive development.

Preferably, the whey-derived composition has an ash content in the range of 0.5-5% w/w relative to total solids, more preferably 1.0-3% w/w, even more preferably 1.5-3% w/w, and most preferably 1.6-2% w/w relative to total solids.

The inventors have found whey-derived compositions having low ash contents to be particularly advantageous for e.g. infant formula products.

In some preferred embodiments of the present invention the whey-derived composition comprises total solids in an amount of 1-30% w/w relative to the weight of the whey-derived composition, more preferably 2-15% w/w, even more preferably 4-12% w/w, and most preferably 5-10% w/w relative to the weight of the whey-derived composition. This is for example useful for whey-derived compositions in the form of liquid products.

Liquid whey-derived composition often comprises total protein in an amount of 0.5-10% w/w relative to the weight of the whey-derived composition, more preferably 1-8% w/w, even more preferably 2-7% w/w, and most preferably 2-5% w/w relative to the weight of the whey-derived composition.

In other preferred embodiments of the present invention, the whey-derived composition comprises total solids in an amount of 90-99% w/w relative to the weight of the whey-derived composition, more preferably 93-98% w/w, even more preferably 94-97% w/w, and most preferably 94-97% w/w relative to the weight of the whey-derived composition. This is e.g. useful for whey-derived compositions in the form of powders or solid products.

The matter of the whey-derived composition that is not solids is preferably water.

The matter of the liquid feed that is not solids is preferably water.

The matter of the filtration retentate that is not solids is preferably water.

The whey-derived composition preferably has a pH in the range of 4.0-8, more preferably 5.5-7.5, even more preferably 5.7-7.0, and most preferably 5.9-6.6.

Typically, the whey-derived composition may comprises at most 10% w/w casein relative to total solids, preferably at most 5% w/w, more preferred at most 1% w/w, and even more preferred at most 0.5% w/w casein relative to the weight of total solids. The whey-derived composition may in some embodiments contain no detectable amount of casein.

Additionally, the whey-derived composition preferably comprise cholesterol. The amount of cholesterol is preferably in the range from 3 to 20 mg/g relative to total solids, more preferably in the range from 4 to 15 mg/g, and most preferably in the range from 5 to 10 mg/g relative to total solids.

The whey-derived composition preferably comprises gangliosides. The most prominent gangliosides of the whey-derived composition are typically GD3 and GM3.

In some preferred embodiments of the present invention, the whey-derived composition comprises GD3 in an amount in the range from 1800 to 3800 mg/kg relative to total solids, most preferably 2000 to 3500 mg/kg relative to total solids.

In some preferred embodiments of the present invention the whey-derived composition comprises GM3 in an amount in the range from 65 to 90 mg/kg relative to total solids, and most preferably in the range from 70 to 85 mg/kg relative to total solids. The total amount of gangliosides of the whey-derived composition may be in the range from 1800 to 4000 mg/kg relative to total solids.

The ganglioside content of the whey-derived composition can be analyzed with a LC-MS method, GANGLIO-r-LC-TOF.

Preferably, the whey-derived composition comprises Immunoglobulin G (IgG, such as IgG1 and IgG2) in the range from 1% w/w to 10% w/w relative to total solids, and more preferably in the range from 3% w/w to 8% w/w relative to total solids. The amount of IgG can be analyzed with radial immunodiffusion.

Preferably, the whey-derived composition comprises bovine serum albumin (BSA). The BSA is preferably present in the amount of 1-5% relative to total solids, and most preferably 2% w/w to 3.5% w/w relative to total solids.

In some embodiments, the whey-derived composition may also comprise glycosylation-dependent cell adhesion molecule (PP3). The PP3 may be present in the amount from 1% w/w to 3.5% w/w relative to the total solids of the whey-derived composition.

In some embodiments, the whey-derived may also comprise lactotransferrin (or lactoferrin). The lactoferrin may be present in the amount from 1% w/w to 1.6% w/w relative the total solids of the whey-derived composition.

The whey-derived composition may further comprise other membrane components.

As used herein, the term “and/or” is intended to mean the combined (“and”) and the exclusive (“or”) use, i.e. “A and/or B” is intended to mean “A alone, or B alone, or A and B together”.

The microbial load of the whey-derived composition is preferably kept to a minimum to make it safe to use in infant product. However, it is a challenge to obtain both a high degree of bioactive of the whey-derived composition and a low content of microorganism as processes for microbial reduction tend to lead to denaturation and degradation of the bioactive components of the whey-derived composition. The present invention makes it possible to obtain a very low content of microorganism while at the same time avoiding to damage the components of the whey-derived composition.

Preferably, the whey-derived composition contains at most 10000 colony-forming units (CFU)/g total solids, more preferably at most 6000 CFU/g total solids, even more preferably at most 3000 CFU/g total solids, and most preferably the whey-derived composition contains at most 1000 CFU/g total solids.

The determination of colony-forming units is based on the total plate count after incubation at 30 degrees C. according to ISO 4833-1.

The inventors have found that even lower contents of microorganisms can be obtained (see e.g. Example 3). Thus in some preferred embodiments of the invention, the whey-derived composition contains at most 600 colony-forming units (CFU)/g total solids, more preferably at most 400 CFU/g total solids, even more preferably at most 200 CFU/g total solids, and most preferably the whey-derived composition contains less than 100 CFU/g total solids.

The inventors have furthermore found that the use of the MF-based germ filtration using a microfiltration membrane having a pore size of 1.0-2 micron, and most preferably 1.2-1.8 micron, in the present method gives rise to a significantly reduced content of endotoxin in the whey-derived composition. The present invention therefore enables the production of phospholipid-enriched whey-derived products containing very low a concentration of endotoxin or even no detectable endotoxin.

In some preferred embodiments of the present invention, the whey-derived composition is a liquid.

In other preferred embodiments of the present invention, the whey-derived composition is a powder, preferably obtained by spray-drying.

In some preferred embodiments of the present invention, the whey-derived composition is a powder comprising:
total lipid in the amount of 10 to 30% w/w relative to total solids,
total phospholipid in an amount of 3 to 12% w/w relative to total solids,
an ash content in the range of 1-10% w/w relative to total solids,
lactose in the amount of at most 10% w/w relative to total solids,
total protein in an amount of 65 to 80% w/w relative to total solids;
optionally, OPN in an amount of 0.8-5% w/w relative to total protein, and
a content of colony forming units (CFU) of at most 1000 CFU/g total solids, and most preferably at most 1000 CFU/g total solids.

In other preferred embodiments of the present invention, the whey-derived composition is a powder comprising:
total lipid in the amount of 10 to 30% w/w relative to total solids,
total phospholipid in an amount of 3 to 12% w/w relative to total solids,
an ash content in the range of 1-10% w/w relative to total solids,
lactose in the amount of at most 10% w/w relative to total solids,
total protein in an amount of 65 to 80% w/w relative to total solids;
optionally, OPN in an amount of 0.8-5% w/w relative to total protein,
a content of colony forming units (CFU) of at most 1000 CFU/g total solids, and most preferably at most 1000 CFU/g total solids, and
a plurality of miRNA species, said plurality of miRNA species comprising at least one miRNA spe-cies selected from the group consisting of let-7a-5p, let-7b, let-7f, let-7i, miR-103, miR16b, miR-191, miR-199a-3p, miR-21-5p, miR-223, miR-26a, miR-26b, miR-423-3p, and miR486.

In further preferred embodiments of the present invention, the whey-derived composition is a powder comprising:
total lipid in the amount of 10 to 30% w/w relative to total solids,
total phospholipid in an amount of 3 to 12% w/w relative to total solids,
an ash content in the range of 1-10% w/w relative to total solids,
lactose in the amount of at most 10% w/w relative to total solids,
total protein in an amount of 65 to 80% w/w relative to total solids,
optionally, OPN in an amount of 0.8-5% w/w relative to total protein,
a content of colony forming units (CFU) of at most 1000 CFU/g total solids, and most preferably at most 1000 CFU/g total solids, and
a plurality of miRNA species, said plurality of miRNA species comprising the miRNA species let-7a-5p, let-7b, let-7f, miR-191, miR-21-5p, and miR-26a.

In some preferred embodiments of the present invention the whey-derived composition of the present invention is obtainable by the method of the present invention.

Yet an aspect of the invention pertains to the use of the whey-derived composition of the invention as a food ingredient for increasing the content of milk extracellular vesicles in a nutritional product, and preferably wherein the nutritional product is a paediatric product and more preferably an infant formula.

A further aspect of the invention pertains to the use of the whey-derived composition of the invention as a food ingredient for increasing the content of miRNA, preferably miRNA present in mammal milk, and most preferably miRNA present in bovine milk and/or in human milk, in a nutritional product, and preferably wherein the nutritional product is a paediatric product and more preferably an infant formula.

The miRNA preferably comprises a plurality of miRNA species, said plurality of miRNA species comprising at least one miRNA species selected from the group consisting of let-7a-5p, let-7b, let-7f, let-7i, miR-103, miR-16b, miR-191, miR-199a-3p, miR-21-5p, miR-223, miR-26a, miR26b, miR-423-3p, and miR-486.

In some preferred embodiments of the invention, the miRNA comprises a plurality of miRNA species, said plurality of miRNA species comprising let-7a-5p, let-7b, let-7f, let-7i, miR-103, miR-16b, miR-191, miR-199a-3p, miR-21-5p, miR-223, miR-26a, miR-26b, miR-423-3p, and miR-486.

Alternatively, but also preferred, the miRNA may comprises a plurality of miRNA species, said plurality of miRNA species comprising at least one miRNA species selected from the group consisting of let-7a-5p, let-7b, let-7f, miR-191, miR-21-5p, and miR-26a.

In other preferred embodiments of the invention the miRNA comprises a plurality of miRNA species, said plurality of miRNA species comprising the miRNA species let-7a-5p, let-7b, let-7f, miR-191, miR-21-5p, and miR-26a.

The whey-derived composition of the invention is preferably used in an amount sufficient to provide a content of solids to the nutritional product of at least 0.1 g/100 g total solids of the nutritional product, more preferably at least 0.5 g/100 g total solids, even more preferably at least 2 g/100 g total solids, and most preferably at least 3 g/100 g total solids.

Preferably, the whey-derived composition is used in an amount sufficient to provide a content of solids to the nutritional product of 0.1-30 g/100 g total solids of the nutritional product, more preferably 0.5-20 g/100 g total solids, even more preferably 2-15 g/100 g total solids, and most preferably 3-12 g/100 g total solids.

An additional aspect of the invention pertains to a nutritional product, which preferably is a paediatric product, and more preferably an infant formula, comprising the whey-derived composition of the invention in an amount sufficient to:
provide OPN in an amount of at least 10 mg/100 g total solids of the nutritional product, more preferably at least 20 mg/100 g total solids, even more preferably at least 30 mg/100 g total solids, and most preferably at least 40 mg/100 g total solids, and/or
provide vitamin B12 in an amount of at least 0.02 microgram/100 g total solids of the nutritional product, more preferably at least 0.05 microgram/100 g total solids, even more preferably at least 0.10 microgram/100 g total solids, and most preferably at least 0.15 microgram/100 g total solids, and/or
to provide a content of solids to the nutritional product of 0.1-30 g/100 g total solids of the nu-tritional product, more preferably 0.5-20 g/100 g total solids, even more preferably 2-15 g/100 g total solids, and most preferably 3-12 g/100 g total solids.

In some preferred embodiments of the invention, the nutritional product, which preferably is a paediatric product, and more preferably an infant formula, comprises the whey-derived composition of the invention in an amount sufficient to:
provide OPN in an amount of at least 10 mg/100 g total solids of the nutritional product, more preferably at least 20 mg/100 g total solids, even more preferably at least 30 mg/100 g total solids, and most preferably at least 40 mg/100 g total solids, and/or
provide vitamin B12 in an amount of at least 0.02 microgram/100 g total solids of the nutritional product, more preferably at least 0.05 microgram/100 g total solids, even more preferably at least 0.10 microgram/100 g total solids, and most preferably at least 0.15 microgram/100 g total solids.

Preferably, the nutritional product comprises the whey-derived composition of the invention in an amount sufficient to provide OPN in an amount of 10-500 mg/100 g total solids of the nutritional product, more preferably 20-400 mg/100 g total solids, even more preferably 30-200 mg/100 g total solids, and most preferably 40-100 mg/100 g total solids.

Preferably, the nutritional product comprises the whey-derived composition of the invention in an amount sufficient to provide vitamin B12 in an amount of 0.02-1.0 microgram/100 g total solids of the nutritional product, more preferably 0.05-0.7 microgram/100 g total solids, even more preferably at least 0.10-0.5 microgram/100 g total solids, and most preferably 0.12-0.4 microgram/100 g total solids.

Preferably, the nutritional product comprises the whey-derived composition of the invention in an amount sufficient to provide a content of solids to the nutritional product of 0.1-30 g/100 g total solids of the nutritional product, more preferably 0.5-20 g/100 g total solids, even more preferably 2-15 g/100 g total solids, and most preferably 3-12 g/100 g total solids.

In the following, preferred numbered embodiments of the invention are described.

Numbered embodiment 1a. A method of preparing a whey-derived composition enriched with respect to phospholipid, and preferably also enriched with respect to other milk fat globule membrane components, the method comprising the steps of:
a) providing a liquid feed comprising whey protein including alpha-lactalbumin (ALA) and phospholipid originating from whey, the liquid feed preferably having a pH in the range of 4.0-8, more preferably 5.5-7.5, even more preferably 5.7-7.0, and most preferably 5.9-6.6.
b) subjecting the liquid feed to membrane filtration to provide a filtration retentate and a filtration permeate, said membrane filtration is arranged and operated to:
   provide a content of total phospholipid relative to total solids of the filtration retentate that is at least 200% of the content of total phospholipid relative to total solids of the liquid feed, and
   provide a content of alpha-lactalbumin on total protein basis of the filtration retentate that is at most 75% of the content of alpha-lactalbumin on total protein basis of the liquid feed, and
c) subjecting the filtration retentate or a product stream comprising at least lipid and protein originating from the filtration retentate to microfiltration using a MF membrane with a pore size in the range of 1.0-2 micrometer and recovering the permeate of the microfiltration, which permeate may be subjected to further processing, and said further processing preferably comprising one or more additional processing steps, preferably comprising one or more of:
   ii) concentration,
   iii) heat-treatment, and
   iv) drying.

Numbered embodiment 1b. A method of preparing a whey-derived composition enriched with respect to phospholipid and osteopontin (OPN), and preferably also enriched with respect to other milk fat globule membrane components, the method comprising the steps of:
a) providing a liquid feed comprising whey protein including osteopontin and alpha-lactalbumin (ALA) and phospholipid originating from whey, the liquid feed containing a total amount of osteopontin in the range of 0.2-2.0% w/w relative to total protein,
b) subjecting the liquid feed to membrane filtration to provide a filtration retentate and a filtration permeate, said membrane filtration is arranged and operated to:
   provide a content of osteopontin on total protein basis of the filtration retentate that is at least 150% of the content of osteopontin on total protein basis of the liquid feed, and
   provide a content of alpha-lactalbumin on total protein basis of the filtration retentate that is at most 75% of the content of alpha-lactalbumin on total protein basis of the liquid feed, and
optionally c) subjecting the filtration retentate or a product stream comprising at least lipid and protein originating from the filtration retentate to one or more additional processing steps, preferably comprising one or more of:
   i) microfiltration,
   ii) concentration,
   iii) heat-treatment, and
   iv) drying.

Numbered embodiment 2. The method according to any of the preceding numbered embodiments wherein the whey of step a) is a sweet whey or an acid whey.

Numbered embodiment 3. The method according to any of the preceding numbered embodiments wherein the liquid feed is a whey.

Numbered embodiment 4. The method according to any of the preceding numbered embodiments wherein the liquid feed is a protein concentrate of a whey.

Numbered embodiment 5. The method according to any of the preceding numbered embodiments wherein the liquid feed is a protein concentrate of the provision of the liquid feed comprises subjecting whey to one or more steps of:
concentration,
demineralisation,
diafiltration, and
at least partial removal of particles having a diameter of 1.5 micrometer or larger.

Numbered embodiment 6. The method according to any of the preceding numbered embodiments wherein the liquid feed comprises total protein in an amount in the range of 5-89% w/w relative to total solids, more preferably 30-86% w/w, even more preferably 40-83% w/w, and most preferably 60-80% w/w relative to total solids.

Numbered embodiment 7. The method according to any of the preceding numbered embodiments wherein the liquid feed comprises total protein in an amount in the range of 5-25% w/w relative to total solids, more preferably 5-20% w/w, even more preferably 5-15% w/w, and most preferably 5-10% w/w relative to total solids.

Numbered embodiment 8. The method according to any of the preceding numbered embodiments wherein the liquid feed comprises a total amount of beta-lactoglobulin in the range of 10-70% w/w relative to total protein, more preferably 30-65% w/w, even more preferably 40-60% w/w, and most preferably 45-55% w/w relative to total protein.

Numbered embodiment 9. The method according to any of the preceding numbered embodiments wherein the liquid feed comprises a total amount of alpha-lactalbumin in the range of 5-40% w/w relative to total protein, more preferably 10-35% w/w, even more preferably 10-30% w/w, and most preferably 10-25% w/w relative to total protein.

Numbered embodiment 10. The method according to any of the preceding numbered embodiments wherein the liquid feed comprises a total amount of caseinomacropeptide in the range of 5-30% w/w relative to total protein, more preferably 10-30% w/w, even more preferably 10-25% w/w, and most preferably 10-20% w/w relative to total protein.

Numbered embodiment 11. The method according to any of the preceding numbered embodiments wherein the liquid feed comprises a total amount of osteopontin in the range of 0.2-0.9% w/w relative to total protein, more preferably 0.3-0.8% w/w, even more preferably 0.4-0.8% w/w, and most preferably 0.4-0.7% w/w relative to total protein.

Numbered embodiment 12. The method according to any of the preceding numbered embodiments wherein the liquid feed comprises a total amount of osteopontin in the range of 1.0-2.0% w/w relative to total protein, more preferably 1.2-2.0% w/w, even more preferably 1.3-2.0% w/w, and most preferably 1.4-2.0% w/w relative to total protein.

Numbered embodiment 13. The method according to any of the preceding numbered embodiments wherein the liquid feed comprises total lipid in an amount in the range of 1-10% w/w relative to total solids, more preferably 2-8% w/w, even more preferably 3-7% w/w, and most preferably 4-7% w/w relative to total solids.

Numbered embodiment 14. The method according to any of the preceding numbered embodiments wherein the liquid feed comprises a total amount of phospholipid in the range of 10-50% w/w relative to total lipid, more preferably 20-47% w/w, even more preferably 25-45% w/w, and most preferably 29-41% w/w relative to total lipid.

Numbered embodiment 15. The method according to any of the preceding numbered embodiments wherein the liquid feed comprises a total amount of phospholipid in the range of 0.2-5% w/w relative to total solids, more preferably 0.4-4% w/w, even more preferably 0.5-3% w/w, and most preferably 1-3% w/w relative to total solids.

Numbered embodiment 16. The method according to any of the preceding numbered embodiments wherein the liquid feed comprises a total amount of free carbohydrate in the range of 0-85% w/w relative to total solids, more preferably 1-55% w/w, even more preferably 1-50% w/w, and most preferably 1-30% w/w relative to total solids.

Numbered embodiment 17. The method according to any of the preceding numbered embodiments wherein the liquid feed comprises a total amount of lactose in the range of 0-80% w/w relative to total solids, more preferably 0-55% w/w, even more preferably 0-50% w/w, and most preferably 0-30% w/w relative to total solids.

Numbered embodiment 18. The method according to any of the preceding numbered embodiments wherein the liquid feed comprises vitamin B12 in an amount in the range of 2-16 microgram/kg total solids, more preferably 4-14 microgram/kg total solids, even more preferably 6-12 microgram/kg total solids, and most preferably 8-10 microgram/kg total solids.

Numbered embodiment 19. The method according to any of the preceding numbered embodiments wherein the liquid feed has an ash content in the range of 1-10% w/w relative to total solids, more preferably 1-8% w/w, even more preferably 2-8% w/w, and most preferably 3-7% w/w relative to total solids.

Numbered embodiment 20. The method according to any of the preceding numbered embodiments wherein the liquid feed comprises total solids in an amount of 1-20% w/w relative to the weight of the liquid feed, more preferably 2-15% w/w, even more preferably 4-12% w/w, and most preferably 5-10% w/w relative to the weight of the liquid feed.

Numbered embodiment 21. The method according to any of the preceding numbered embodiments wherein the liquid feed comprises total protein in an amount of 0.2-8% w/w relative to the weight of the liquid feed, more preferably 1-7% w/w, even more preferably 2-6% w/w, and most preferably 2-5% w/w relative to the weight of the liquid feed.

Numbered embodiment 22. The method according to any of the preceding numbered embodiments wherein the liquid feed has a pH in the range of 4.0-8, more preferably 5.5-7.5, even more preferably 5.7-7.0, and most preferably 5.9-6.6.

Numbered embodiment 23. The method according to any of the preceding numbered embodiments wherein the membrane filtration of step b) is arranged and operated to provide a content of beta-lactoglobulin on total protein basis of the filtration retentate that is at most 100% of the content of beta-lactoglobulin on total protein basis of the liquid feed, more preferably at most 90%, even more preferred at most 80% and most preferred at most 70% of the content of beta-lactoglobulin on total protein basis of the liquid feed.

Numbered embodiment 24. The method according to any of the preceding numbered embodiments wherein the membrane filtration of step b) is arranged and operated to provide a content of beta-lactoglobulin on total protein basis of the filtration retentate that is in the range of 10-100% of the content of beta-lactoglobulin on total protein basis of the liquid feed, more preferably 20-98%, even more preferred 30-96% and most preferred 40-94% of the content of betalactoglobulin on total protein basis of the liquid feed.

Numbered embodiment 25. The method according to any of the preceding numbered embodiments wherein the membrane filtration of step b) is arranged and operated to provide a content of beta-lactoglobulin on total protein basis of the filtration retentate that is in the range of 10-90% of the content of beta-lactoglobulin on total protein basis of the liquid feed, more preferably 15-80%, even more preferred 20-70% and most preferred 25-60% of the content of betalactoglobulin on total protein basis of the liquid feed.

Numbered embodiment 26. The method according to any of the preceding numbered embodiments wherein the membrane filtration of step b) is arranged and operated to provide a content of alpha-lactalbumin on total protein basis of the filtration retentate that is at most 50% of the content of alpha-lactalbumin on total protein basis of the liquid feed, more preferably at most 30%, even more preferred at most 20% and most preferred at most 10% of the content of alpha-lactalbumin on total protein basis of the liquid feed.

Numbered embodiment 27. The method according to any of the preceding numbered embodiments wherein the membrane filtration of step b) is arranged and operated to provide a content of alpha-lactalbumin on total protein basis of the filtration retentate that is in the range of 1-50% of the content of alpha-lactalbumin on total protein basis of the liquid feed, more preferably 2-30%, even more preferred 3-20% and most preferred 4-10% of the content of alpha-lactalbumin on total protein basis of the liquid feed.

Numbered embodiment 28. The method according to any of the preceding numbered embodiments wherein the membrane filtration of step b) is arranged and operated to provide a content of alpha-lactalbumin on total protein basis of the filtration retentate that is in the range of 5-50% of the content of alpha-lactalbumin on total protein basis of the liquid feed, more preferably 10-45%, even more preferred 15-40% and most preferred 20-35% of the content of alpha-lactalbumin on total protein basis of the liquid feed.

Numbered embodiment 29. The method according to any of the preceding numbered embodiments wherein the membrane filtration of step b) is arranged and operated to provide a content of caseinomacropeptide on total protein basis of the filtration retentate that is at most 50% of the content of caseinomacropeptide on total protein basis of the liquid feed, more preferably at most 40%, even more preferred at most 35% and most preferred at most 30% of the content of caseinomacropeptide on total protein basis of the liquid feed.

Numbered embodiment 30. The method according to any of the preceding numbered embodiments wherein the membrane filtration of step b) is arranged and operated to provide a content of caseinomacropeptide on total protein basis of the filtration retentate that is in the range of 1-50% of the content of caseinomacropeptide on total protein basis of the liquid feed, more preferably 2-40%, even more preferred 3-35% and most preferred 4-30% of the content of caseinomacropeptide on total protein basis of the liquid feed.

Numbered embodiment 31. The method according to any of the preceding numbered embodiments wherein the membrane filtration of step b) is arranged and operated to provide a content of caseinomacropeptide on total protein basis of the filtration retentate that is in the range of 1-45% of the content of caseinomacropeptide on total protein basis of the liquid feed, more preferably 2-30%, even more preferred 3-20% and most preferred 4-10% of the content of caseinomacropeptide on total protein basis of the liquid feed.

Numbered embodiment 32. The method according to any of the preceding numbered embodiments wherein the membrane filtration of step b) is arranged and operated to provide a content of osteopontin on total protein basis of the filtration retentate that is at least 180% of the content of osteopontin on total protein basis of the liquid feed, more preferably at least 200%, even more preferably at least 230%, and most preferably at least 250% of the content of osteopontin on total protein basis of the liquid feed.

Numbered embodiment 33. The method according to any of the preceding numbered embodiments wherein the membrane filtration of step b) is arranged and operated to provide a content of osteopontin on total protein basis of the filtration retentate that is in the range of 150-600% of the content of osteopontin on total protein basis of the liquid feed, more preferably 175-500%, even more preferably 200-450%, and most preferably 225-300% of the content of osteopontin on total protein basis of the liquid feed.

Numbered embodiment 34. The method according to any of the preceding numbered embodiments wherein the membrane filtration of step b) is arranged and operated to provide a content of total phospholipid relative to total solids of the filtration retentate that is at least 200% of the content of total phospholipid relative to total solids of the liquid feed, more preferably at least 225%, even more preferably at least 250%, and most preferably at least 275% of the content of total phospholipid relative to total solids of the liquid feed.

Numbered embodiment 35. The method according to any of the preceding numbered embodiments wherein the membrane filtration of step b) is arranged and operated to provide a content of total phospholipid relative to total solids of the filtration retentate that is in the range of 200-600% of the content of total phospholipid relative to total solids of the liquid feed, more preferably 225-550%, even more preferably 250-500%, and most preferably 275-450% of the content of total phospholipid relative to total solids of the liquid feed.

Numbered embodiment 36. The method according to any of the preceding numbered embodiments wherein the membrane filtration of step b) involves one or more membrane(s) with a nominal molecular weight cut-off in the range of 100-2000 kDa, more preferably 300-1600 kDa; even more preferably 500-1300 kDa, and most preferably 700-1000 kDa.

Numbered embodiment 37. The method according to any of the preceding numbered embodiments wherein the membrane filtration of step b) involves diafiltration.

Numbered embodiment 38. The method according to any of the preceding numbered embodiments wherein the intermediate retentate stream(s) during step b), if any, comprise total protein in an amount of 0.5-10% w/w relative to the weight of the intermediate retentate stream, more preferably 1-8% w/w, even more preferably 2-7% w/w, and most preferably 2-5% w/w relative to the weight of the intermediate retentate stream.

Numbered embodiment 39. The method according to any of the preceding numbered embodiments wherein the membrane filtration of step b) is operated with a trans-membrane pressure of 0.1-5 bar, more preferably 0.2-3 bar and most preferably 0.3-1 bar.

Numbered embodiment 40. The method according to any of the preceding numbered embodiments wherein the membrane filtration of step b) is operated at a temperature of 1-60 degrees C., more preferably 2-30 degrees C., even more preferably 5-20 degrees C., and most preferably 8-15 degrees C.

Numbered embodiment 41. The method according to any of the preceding numbered embodiments wherein the method furthermore comprises step c).

Numbered embodiment 42. The method according to any of the preceding numbered embodiments wherein the method furthermore comprises step c), and step c) comprises i) microfiltration, preferably microfiltering the filtration retentate or a product stream comprising at least lipid and protein originating from the filtration retentate, preferably using a MF membrane with a pore size in the range of 1.0-2 micrometer.

Numbered embodiment 43. The method according to any of the preceding numbered embodiments wherein the method furthermore comprises step c), and step c) comprises ii) concentration, preferably using one or more of ultrafiltration, nanofiltration, reverse osmosis, and evaporation.

Numbered embodiment 44. The method according to any of the preceding numbered embodiments wherein the method furthermore comprises step c), and step c) comprises iii) heat-treated, preferably comprising heat-treatment to a temperature of at least 60 degrees C. for a duration sufficient for obtaining at least partial microbial reduction.

Numbered embodiment 45. The method according to any of the preceding numbered embodiments wherein the method furthermore comprises step c), and step c) comprises iv) drying, preferably comprising or even consisting of spray-drying.

Numbered embodiment 46. The method according to any of the preceding numbered embodiments wherein the method furthermore comprises step c), and step c) comprises subjecting the filtration retentate or a product stream comprising at least lipid and protein originating from the filtration retentate to:
i) microfiltration, preferably using a MF membrane with a pore size in the range of 1.0-2 micrometer, followed by
ii) concentration, preferably using ultrafiltration, nanofiltration and/or reverse osmosis, followed by
iii) heat-treatment, preferably involving heating the liquid to be heat-treated to a temperature of at least 60 degrees C. for a duration sufficient for obtaining at least partial microbial reduction, but wherein the heat-treatment denatures at most 5% of the BLG of the liquid to be heat-treated, and followed by
iv) drying, preferably comprising or even consisting of spray-drying. 47. A whey-derived composition comprising:
total lipid in the amount of 10 to 30% w/w relative to total solids,
total phospholipid in an amount of 3 to 12% w/w relative to total solids,
an ash content in the range of 1-10% w/w relative to total solids,
lactose in the amount of at most 10% w/w relative to total solids,
total protein in an amount of 65 to 80% w/w relative to total solids,
a content of colony forming units (CFU) of at most 10000 CFU/g total solids.

Numbered embodiment 47a. A whey-derived composition comprising:
total lipid in the amount of 10 to 30% w/w relative to total solids,
total phospholipid in an amount of 3 to 12% w/w relative to total solids,
an ash content in the range of 1-10% w/w relative to total solids,
lactose in the amount of at most 10% w/w relative to total solids;
total protein in an amount of 65 to 80% w/w relative to total solids,
OPN in an amount of 0.8-5% w/w relative to total protein.

Numbered embodiment 48. The whey-derived composition according to numbered embodiments 47 or 47a wherein the whey-derived composition comprises total protein in an amount in the range of 66-78% w/w relative to total solids, more preferably 68-76% w/w, and most preferably 70-76% w/w relative to total solids.

Numbered embodiment 49. The whey-derived composition according to any of the numbered embodiments 47-48 wherein the whey-derived composition comprises a total amount of betalactoglobulin in the range of 10-45% w/w relative to total protein, more preferably 15-40% w/w, even more preferably 20-40% w/w, and most preferably 25-35% w/w relative to total protein.

Numbered embodiment 50. The whey-derived composition according to any of the numbered embodiments 47-49 wherein the whey-derived composition comprises a total amount of alpha-lactalbumin in the range of 0-10% w/w relative to total protein, more preferably 0.1-8% w/w, even more preferably 0.3-5% w/w, and most preferably 0.5-3% w/w relative to total protein.

Numbered embodiment 51. The whey-derived composition according to any of the numbered embodiments 47-50 wherein the whey-derived composition comprises a total amount of alpha-lactalbumin in the range of 1-10% w/w relative to total protein, more preferably 1-9% w/w, even more preferably 2-8% w/w, and most preferably 3-7% w/w relative to total protein.

Numbered embodiment 52. The whey-derived composition according to any of the numbered embodiments 47-51 wherein the whey-derived composition comprises a total amount of caseinomacropeptide in the range of 0-10% w/w relative to total protein, more preferably 1-8% w/w, even more preferably 2-7% w/w, and most preferably 3-7% w/w relative to total protein.

Numbered embodiment 53. The whey-derived composition according to any of the numbered embodiments 47-52 wherein the whey-derived composition comprises a total amount of caseinomacropeptide in the range of 0-9% w/w relative to total protein, more preferably 0.1-7% w/w, even more preferably 0.3-5% w/w, and most preferably 0.5-3% w/w relative to total protein.

Numbered embodiment 54. The whey-derived composition according to any of the numbered embodiments 47-53 wherein the whey-derived composition comprises a total amount of osteopontin in the range of 0.9-5% w/w relative to total protein, more preferably 1.0-4% w/w, even more preferably 1.1-3% w/w, and most preferably 1.1-1.7% w/w relative to total protein.

Numbered embodiment 55. The whey-derived composition according to any of the numbered embodiments 47-54 wherein the whey-derived composition comprises a total amount of osteopontin in the range of 2.0-5% w/w relative to total protein, more preferably 2.2-4.5% w/w, even more preferably 2.5-4.0% w/w, and most preferably 3.0-3.7% w/w relative to total protein.

Numbered embodiment 56. The whey-derived composition according to any of the numbered embodiments 47-55 wherein the whey-derived composition comprises a total lipid in an amount in the range of 10-29% w/w relative to total solids, more preferably 11-27% w/w, even more preferably 13-25% w/w, and most preferably 16-22% w/w relative to total solids.

Numbered embodiment 57. The whey-derived composition according to any of the numbered embodiments 47-56 wherein the whey-derived composition comprises a total amount of phospholipid in the range of 10-50% w/w relative to total lipid, more preferably 20-47% w/w, even more preferably 25-45% w/w, and most preferably 29-41% w/w relative to total lipid.

Numbered embodiment 58. The whey-derived composition according to any of the numbered embodiments 47-57 wherein the whey-derived composition comprises a total amount of phospholipid in the range of 4-12% w/w relative to total solids, more preferably 4-11% w/w, even more preferably 5-11% w/w, and most preferably 6-10% w/w relative to total solids.

Numbered embodiment 59. The whey-derived composition according to any of the numbered embodiments 47-58 wherein the whey-derived composition comprises a total amount of free carbohydrate in the range of 0-8% w/w relative to total solids, more preferably 0-5% w/w, even more preferably 0-1% w/w, and most preferably 0-0.5% w/w relative to total solids.

Numbered embodiment 60. The whey-derived composition according to any of the numbered embodiments 47-59 wherein the whey-derived composition comprises a total amount of lactose in the range of 0-8% w/w relative to total solids, more preferably 0-5% w/w, even more preferably 0-1% w/w, and most preferably 0-0.5% w/w relative to total solids.

Numbered embodiment 61. The whey-derived composition according to any of numbered embodiments 47-60 wherein the whey-derived composition comprises vitamin B12 in an amount in the range of 20-60 microgram/kg total solids, more preferably 24-50 microgram/kg total solids even more preferably 26-45 microgram/kg total solids, and most preferably 30-40 microgram/kg total solids.

Numbered embodiment 62. The whey-derived composition according to any of the numbered embodiments 47-61 wherein the whey-derived composition has an ash content in the range of 0.5-5% w/w relative to total solids, more preferably 1.0-3% w/w, even more preferably 1.5-3% w/w, and most preferably 1.6-2% w/w relative to total solids.

Numbered embodiment 63. The whey-derived composition according to any of the numbered embodiments 47-62 wherein the whey-derived composition comprises total solids in an amount of 1-30% w/w relative to the weight of the whey-derived composition, more preferably 2-15% w/w, even more preferably 4-12% w/w, and most preferably 5-10% w/w relative to the weight of the whey-derived composition.

Numbered embodiment 64. The whey-derived composition according to numbered embodiment 63 wherein the whey-derived composition comprises total protein in an amount of 0.5-10% w/w relative to the weight of the whey-derived composition, more preferably 1-8% w/w, even more preferably 2-7% w/w, and most preferably 2-5% w/w relative to the weight of the whey-derived composition.

Numbered embodiment 65. The whey-derived composition according to any of the numbered embodiments 47-64 wherein the whey-derived composition comprises total solids in an amount of 90-99% w/w relative to the weight of the whey-derived composition, more preferably 93-98% w/w, even more preferably 94-97% w/w, and most preferably 94-97% w/w relative to the weight of the whey-derived composition.

Numbered embodiment 66. The whey-derived composition according to any of the numbered embodiments 47-65 wherein the whey-derived composition has a pH in the range of 4.0-8, more preferably 5.5-7.5, even more preferably 5.7-7.0, and most preferably 5.9-6.6.

Numbered embodiment 67. The whey-derived composition according to any of the numbered embodiments 47-66 wherein the whey-derived composition is a liquid or a powder.

Numbered embodiment 68. The whey-derived composition according to any of the numbered embodiments 47-67 obtainable by one or more of numbered embodiments 1-46.

Numbered embodiment 69. Use of the whey-derived composition according to any of the numbered embodiments 47-68 as a food ingredient, preferably for increasing the content of OPN in a nutritional product, and preferably wherein the nutritional product is a paediatric product and more preferably an infant formula; preferably using the whey-derived composition in an amount sufficient to provide a content of OPN of to the nutritional product of at least 10 mg/100 g total solids of the nutritional product, more preferably at least 20 mg/100 g total solids, even more preferably at least 30 mg/100 g total solids, and most preferably at least 40 mg/100 g total solids.

Numbered embodiment 70. The use according to numbered embodiment 69 wherein the whey-derived composition is used in an amount sufficient to provide a content of OPN to the nutritional product of 10-500 mg/100 g total solids of the nutritional product, more preferably 20-400 mg/100 g total solids, even more preferably 30-200 mg/100 g total solids, and most preferably 40-100 mg/100 g total solids.

Numbered embodiment 71. Use of the whey-derived composition according to any of the numbered embodiments 47-68 as a food ingredient, preferably for increasing the content of vitamin B12 in a nutritional product, and preferably wherein the nutritional product is a paediatric product and more preferably an infant formula; preferably using the whey-derived composition in an amount sufficient to provide a content of vitamin B12 to the nutritional product of at least 0.02 microgram/100 g total solids of the nutritional product, more preferably at least 0.05 microgram/100 g total solids, even more preferably at least 0.10 microgram/100 g total solids, and most preferably at least 0.15 microgram/100 g total solids.

Numbered embodiment 72. Use of the whey-derived composition according to numbered embodiment 71 wherein the whey-derived composition is used in an amount sufficient to provide a content of vitamin B12 to the nutritional product of 0.02-1.0 microgram/100 g total solids of the nutritional product, more preferably 0.05-0.7 microgram/100 g total solids, even more preferably at least 0.10-0.5 microgram/100 g total solids, and most preferably 0.12-0.4 microgram/100 g total solids.

Numbered embodiment 73. A nutritional product, which preferably is a paediatric product, and more preferably an infant formula, comprising the whey-derived composition according to any of the numbered embodiments 47-68 in an amount sufficient to:
provide OPN in an amount of at least 10 mg/100 g total solids of the nutritional product, more preferably at least 20 mg/100 g total solids, even more preferably at least 30 mg/100 g total solids, and most preferably at least 40 mg/100 g total solids, and/or
provide vitamin B12 in an amount of at least 0.02 microgram/100 g total solids of the nutritional product, more preferably at least 0.05 microgram/100 g total solids, even more preferably at least 0.10 microgram/100 g total solids, and most preferably at least 0.15 microgram/100 g total solids, and/or
provide a content of solids to the nutritional product of 0.1-30 g/100 g total solids of the nutritional product, more preferably 0.5-20 g/100 g total solids, even more preferably 2-15 g/100 g total solids, and most preferably 3-12 g/100 g total solids.

Numbered embodiment 74. The nutritional product according to numbered embodiment 73, comprising the whey-derived composition according to any of the numbered embodiments 47-68 in an amount sufficient to provide OPN in an amount of 10-500 mg/100 g total solids of the nutritional product, more preferably 20-400 mg/100 g total solids, even more preferably 30-200 mg/100 g total solids, and most preferably 40-100 mg/100 g total solids.

Numbered embodiment 75. The nutritional product according to numbered embodiment 73 or 74, comprising the whey-derived composition according to any of the numbered embodiments 47-68 in an amount sufficient to provide vitamin B12 in an amount of 0.02-1.0 microgram/100 g total solids of the nutritional product, more preferably 0.05-0.7 microgram/100 g total solids, even more preferably at least 0.10-0.5 microgram/100 g total solids, and most preferably 0.12-0.4 microgram/100 g total solids.

Numbered embodiment 75. The nutritional product according to numbered embodiment 73 or 74, comprising the whey-derived composition according to any of the numbered embodiments 47-68 in an amount sufficient to provide vitamin a content of solids to the nutritional product of 0.1-30 g/100 g total solids of the nutritional product, more preferably 0.5-20 g/100 g total solids, even more preferably 2-15 g/100 g total solids, and most preferably 3-12 g/100 g total solids.

Numbered embodiment 76. The nutritional product according to any of the numbered embodiment 73-75, comprising the whey-derived composition according to any of the numbered embodiments 47-68 in an amount sufficient to provide a content of solids to the nutritional product of 0.1-30 g/100 g total solids of the nutri-tional product, more preferably 0.5-20 g/100 g total solids, even more preferably 2-15 g/100 g total solids, and most preferably 3-12 g/100 g total solids.

### Examples

### Analyses

### Analysis 1—Quantification of OPN Including Full Length OPN and Naturally Occurring Long OPN Fragments

### Analytical Principle:

A liquid sample is filtered through a 0.22 μm filter and subjected to HPLC with the anionic exchange column MonoQ HR 5/5 (1 ml), Pharmacia, and detection at 280 nm. The concentration of the sample is calculated by the external standard method (comparison with peak area of standard with known OPN content). It has been confirmed that both full length OPN and the naturally occurring long OPN fragments of milk or whey elute in the same peak using the present method.
Reagents: OPN standard, Milli Q water, HPLC grade, NaCl, Merck, Tris HCl, Sigma
Buffer A: 10 mM NaCl, 20 mM Tris HCL, pH 8.0
Buffer B: 0.8 M NaCl, 20 mM Tris HCl, pH 8.0

A standard calibration curve was made from 5 standards in the concentration range 1-10 mg/ml of OPN standard in buffer A. All standards were filtered by 0.22 μm filters before loading onto the column.

### Sampling and Pre-Treatment Pretreatment:

Powder samples are initially converted to liquid samples by dissolving the powder samples in Milli Q water. The liquid samples for analysis are diluted with Milli Q water, HPLC grade, if they are out of range of the standard calibration curve. Dilution is in some instances also necessary to enable binding of OPN to the anion exchange resin if much NaCl from the eluent is present. An amount equivalent to 25 μL of 1-10 mg/mL OPN is injected for analysis. Samples are filtered through 0.22 μm filters before injection to HPLC.

HPLC conditions: Flow 1 ml/min, injection volume 25 μL, gradient: 0-3 min 0% B, 3-17 min 0-60% B, 17-30 min 60-100% B, 30-33 min 100% B, 33-34 min 100-0% B, 34-40 min 0% B.

### Calculation and Expression of Results:

The concentration of OPN in each sample is calculated by reference to the standard curve and by observing the employed dilutions. The weight percentage of OPN relative to total protein or total solids furthermore requires that the content of total protein or total solids of the sample is determined.

### Analysis 2—Determination of the Percentage of EV-Derived Phospholipid Relative to Total Phospholipid

### Basis of the Analysis:

The phospholipid pool found in milk/whey is mainly made up of material originating from extracellular vesicles and the milk fat globule membrane. As these two phospholipid membranes have different biological origin, they can be distinguished by their protein cargo. An integral membrane protein that is a good (but non-unique) marker for MFGM is Butyrophilin, and the tetraspanin CD9 is a good non-unique integral membrane protein marker for extracellular vesicle material. Quantifying these two proteins and evaluating the molar ratio between them becomes a good dimensionless measure of the ratio between EV and MFGM material in the phospholipid source. Using pure EV and MFGM reference material (Blans et al; Pellet-free isolation of human and bovine milk extracellular vesicles by size-exclusion chromatography; 2017; Journal of Extracellular Vesicles, 6:1, DOI: 10.1080/20013078.2017.1294340), this BTN/CD9 molar ratio can directly be converted to a standard curve showing the percentage of EV and MFGM material that makes up the total phospholipid pool. One of the main advantages of this analysis design is that it does not require supporting measurements like the concentration of dry-matter/protein/phospholipid etc., thereby making it less vulnerable to accumulating experimental errors and fully independent of the physical state of the sample.

### Preparation of Standards:

Non-labelled synthetic peptides were purchased from Thermo Fisher Scientific GmbH (Ulm, Germany) as powder and dissolved in Milli-Q purified water (Milli-Q academic, Merck Millipore) according to the manufactures' instructions to reach a concentration of 50 μM. Aliquots were stored at −20° C. until further use.

### Preparation of Samples:

Samples were diluted in 50 mM TEAB (50 mM tetraethylammonium bromide (TEAB) pH 8.5) to a protein concentration of 1.5 mg·mL-1. Samples with lower protein concentration were treated accordingly. All powder samples were left to solubilize over night at 4° C. 100 μL 1.5 mg·mL-1 protein sample in 50 mM TEAB was mixed with 40 μL 100 mM DTT. The sample was reduced at 100° C. for 30 min. After reduction, 140 μL 100 mM iodoacetamide was added. The sample was amidated for 30 min at room temperature screened from light. After amidation, 25 μL 0.3 μg·μL⁻¹ trypsin (TPCK treated, bovine pancreas, 10,000 BAEE units/mg protein, T1426, Sigma Aldrich) was added to the sample and subsequently diluted with 180 μL 50 mM TEAB. The sample was digested at 37° C. for 20 hours. Trypsin was inactivated by lowering pH to 3 with 15 μL 10% TFA to a final concentration of 0.3% TFA. Final concentration of protein is 0.3 mg·mL-1 and the volume is 500 μL.

### RP-HPLC-ESI-QQQ:

Separation of tryptic peptides was performed at 45° C. on an Agilent 1200 Series system (Agilent Technologies) equipped with a RP Symmetry300™ C18-column (5 μm, 2.1×150 mm, Waters Corp.) and a guard column Sentry RP Symmetry300™ C18-column (3.5 μm, 2.1×5 mm, Waters Corp.). Injection volume was 25 μL. Separation is achieved with the following gradient at a flow of 0.35 mL·min⁻¹:

| | |
|---|---|
| Mobile phase A | 0.1% (v/v) Formic acid in Milli-Q purified water |
| | (Milli-Q academic, Merck Millipore) |
| Mobile phase B | 0.1% (v/v) Formic acid in acetonitrile |
| | (HPLC grade S, Rathburn) |

| Time | Mobile phase B [%] |
|---|---|
| 0 | |
| 2 | 3 |
| 25 | 29.4 |
| 35 | 100 |
| 39 | 100 |
| 40 | 3 |
| 45 | 3 |

MS detection were performed on an Agilent 6410 Triple-Quad LC/MS (Agilent Technologies) in positive ESI mode at the following conditions:

| Setting | Value | |
|---|---|---|
| Gas temperature | 300° | C. |
| Gas flow | 10 | L · min⁻¹ |
| Nebulizer gas pressure | 50 | psi |
| Capillary voltage | 4000 | V |
| Nozzle voltage 600 V | 600 | V |
| Cell accelerator voltage | 7 | V |

MS analysis was performed in Single reaction monitoring mode, time segments were applied as far as the chromatographic resolution allowed it. UV spectra were recorded at 214 nm. Data processing was done with MassHunter Quantitative Analysis Software (B.06.00, Agilent Technologies).

Peptide used for detecting Butyrophilin (Protein ID: P18892) was TPLPLAGPPR and the peptide for CD9 (Protein ID: P30932) was NLIDSLK.

### Preparing the Standard Curve:

Using Butyrophilin (BTN) as a non-unique marker for MFGM material and CD9 as a non-unique marker for EVs, pure samples of EVs and MFGM were analysed for both proteins and linear extrapolations were made for both proteins and used for calculating a standard curve showing the calculated content of EV-derived phospholipid relative total phospholipid vs. the molar ratio between BTN and CD9 (i.e. BTN/CD9). The resulting standard curve is shown in FIG. 1.

### Quantification of the Percentage of EV-Derived Phospholipid Relative to Total Phospholipid:

The percentage of EV-derived phospholipid relative total phospholipid of a whey-derived product is determined by measuring the contents of BTN and CD9 of a sample of the whey-derived product using the procedure described above and by calculating the molar ratio between BTN and CD9 (i.e. BTN/CD9) of the sample. The molar ratio is then compared to the standard curve to determine the corresponding percentage of EV-derived phospholipid relative to total phospholipid.

### Example 1—Production of a Whey-Derived Composition Enriched in Milk Fat Globular Membrane Components

A whey-derived powder enriched with respect to whey phospholipids and OPN was prepared in the following manner.

A whey protein concentrate (WPC70) was prepared by ultrafiltration of a bovine sweet whey from cheese production until 70% protein of total solids was obtained. The ultrafiltration membrane had a nominal molecular weight cut-off of 5 kDa and the operating temperature was approx. 15 degrees C.

The WPC70 was then subjected to membrane filtration using Synder FR membrane (nominal molecular weight cut-off of 800 kDa; spiral-wound element with a polymeric membrane (polyvinylidene difluoride-based)) using diafiltration with water as diluent, a trans-membrane pressure of approx. 0.5 bar and a process temperature of approx. 10 degrees C. The membrane filtration was continued until ALA content of the retentate had been reduced to approx. 33% of the initial ALA content of the WPC70.

Approximately 1200 kg of the final filtration retentate was collected and a sample of the retentate was analysed (see Table 1).

1000 kg of the collected filtration retentate was diluted to 4600 kg with water and subjected to microbial reduction (germ filtration) using microfiltration with TAMI 1.4 micrometer Isoflux ceramic elements and diafiltration to wash whey protein, phospholipids and other bioactive components in the MF permeate. The germ filtration was operated at 15 degrees C. The MF permeate was subsequently concentrated by reverse osmosis at 15 degrees C., heat-treated to a temperature of 66° C. and held at this temperature for 15 seconds, and finally spray-dried. A sample of the whey-derived powder was analysed and its chemical composition is shown in Table 1.

TABLE 1
Composition of the liquid feed (WPC70), the filtration retentate,
and the phospholipid-enriched whey-derived powder. The weight
percentages are relative to total solids of the mentioned
compositions except for the weight percentages of the protein
species which are provided relative to total protein.

| | | | | Phospholipid |
|---|---|---|---|---|
| | | | Filtra- | enriched, |
| | | | tion | whey-derived |
| | Unit | WPC70 | retentate | powder |
| Total | % w/w TS | 1.8 | 7.9 | 8.3 |
| phospholipid | | | | |
| Total lipid | % w/w TS | 6 | 20.6 | 19.2 |
| Total protein | % w/w TS | 73 | 74.3 | 75.4 |
| IgG | % w/w TOP | 4.5 | 8.5 | 8.4 |
| Beta- | % w/w TOP | 47 | 41 | 41 |
| lactoglobulin | | | | |
| Alpha- | % w/w TOP | 18 | 6 | 6 |
| lactalbumin | | | | |
| Osteopontin | % w/w TOP | 0.6 | 1.5 | 1.5 |
| Caseinomacro- | % w/w TOP | 17 | 4 | 4 |
| peptide | | | | |
| Lactose | % w/w TS | 15 | 0.94 | 0.65 |
| Sialic Acid | % w/w TS | 1.0 | 2.1 | 2.1 |
| Ash content | % w/w TS | 3.3 | 2.0 | 2.2 |
| B12 | Microgram/kg | 9 | 33 | 33 |
| | TS | | | |
| Na | % w/w TS | 0.20 | 0.05 | 0.11 |
| Mg | % w/w TS | 0.07 | 0.05 | 0.05 |
| P | % w/w TS | 0.36 | 0.45 | 0.45 |
| Cl | % w/w TS | 0.13 | 0.04 | 0.04 |
| K | % w/w TS | 0.77 | 0.20 | 0.33 |
| Ca | % w/w TS | 0.43 | 0.31 | 0.31 |

The whey-derived powder contained significantly less than 10000 colony-forming units/g of total solids.

### Conclusion

The inventors confirmed their initial finding that germ filtration with a controlled pore size can be used to reduce the microbial load of the product without large changes in the composition of whey derived powder relative to the filtration retentate. Additionally the inventors found that the combination of the germ filtration with gentle heat-treatment provided a whey derived powder with a very low microbial load. These discoveries are characterised in further detail in Example 3.

The inventors furthermore found that the product streams of the present method surprisingly may contain a substantial amount of milk-derived extracellular vesicles and microRNA. These discoveries are described in further detail in Examples 4 and 5.

The inventors additionally found that surprisingly both osteopontin (OPN) and vitamin B12 were enriched together with the whey phospholipids during the membrane filtration of Example 1 contrary to what was expected previously. The invention therefore enables the production of improved whey phospholipid products which have an increased content of OPN and vitamin B12. This is particularly advantageous in relation to infant nutrition as both OPN and vitamin B12 are important components for infant development.

OPN is an important nutritional component for the development of the infant immune system and nervous tissue and supplements e.g. sialic acid, immunoglobulins, and complex whey lipids such as whey phospholipids and gangliosides which are also found and enriched in the whey-derived powder.

Vitamin B12 is important for the cognitive development of infants and supplement the abovementioned bioactive components which also have been found to contribute to the development of infant cognition. The inventors have furthermore seen indications that the present whey-derived B12 is particularly useful for infant nutrition as the vitamin seems to be associated to other bioactive components in the whey-derived powder and may have a better bioavailability.

### Example 2—Production of an Infant Formula Comprising the High OPN, Whey-Derived Composition

Two samples of infant formula (IF) powder were prepared by thoroughly mixing the ingredients described in the table below. The sample “WDP IF powder” contained the novel whey-derived powder (WDP) of Example 1 in addition to a whey protein concentrate powder (80% protein), palm oil, lactose, and skim milk powder, whereas the sample “Reference IF powder” only contained the traditional IF components.

| | | WDP IF | Reference |
|---|---|---|---|
| Ingredients | Unit | powder | IF powder |
| Skimmed Milk Powder (96.8% total | % w/w | 14.0 | 14.0 |
| solids) | | | |
| Palm Oil | % w/w | 25.1 | 25.7 |
| Premium Lactose | % w/w | 53.8 | 53.5 |
| Novel whey-derived powder of | % w/w | 4.7 | |
| Example 1 | | | |
| WPC80 powder (prepared from sweet | % w/w | 2.4 | 6.7 |
| whey by ultra-filtration; 80% w/w | | | |
| protein) | | | |
| Selected characteristics | | | |
| Whey protein:casein weight ratio | | 60:40 | 60:40 |
| Energy content | kcal/100 g | 510 | 510 |
| Protein (N*6.25) | g/100 kcal | 2 | 2 |
| OPN (N*6.25) | mg/100 g | 74 | 43 |
| OPN (N*6.25) when ready to drink | mg/L | 95 | 55 |
| (129 g IF formula powder/L)) | | | |
| Total phospholipid | g/100 g | 365 | 101 |
| Total phospholipid when ready to | mg/L | 471 | 130 |
| drink (129 IF formula powder g/L) | | | |

The “ready to drink” values are based on a mixture of the IF powder samples and water in sufficient amounts to get 129 g IF powder per 1 L final drink.

### Example 3—Further Investigations of the Process and Products of Example 1

The process described in Example 1 was further investigated to quantify the loss of nutrients during the MF-based germ filtration. A large-scale implementation of Example 1 was furthermore tested to further characterize the impact of MF-based germ filtration on the microbial quality of the resulting whey-derived products.

### Loss of Valuable Nutrients Due to Germ Filtration

The mass balance was calculated over the MF-based germ filtration step for two batches run according to Example 1 to assess to which extent valuable nutrients such as protein and phospholipids were lost to the retentate stream of the germ filtration. The results are summarised in Table 2.

TABLE 2
Loss of protein, phospholipids and fat due to the
MF-based germ filtration. Calculated on two batches
(Batch A and B) produced according to Example 1.

| | | |
|---|---|---|
| | Recovered in the | |
| In the feed | retentate stream | |
| for the germ | of the germ | % lost in the |
| filtration | filtration | retentate stream |

Batch A

| | | | |
|---|---|---|---|
| Protein (kg) | 194.4 | 6.03 | 3.1% |
| Phospholipid | 25.2 | 0.31 | 1.2% |
| (kg) | | | |
| Fat (kg) | n.d. | n.d. | n.d. |

Batch B

| | | | |
|---|---|---|---|
| Protein (kg) | 303.5 | 5.76 | 1.9% |
| Phospholipid | 20.8 | 0.22 | 1.1% |
| (kg) | | | |
| Fat (kg) | 57.0 | 4.19 | 7.3% |

The inventors were surprised to find that only approx. 1% of the phospholipids and only 2-3% of the protein were lost whereas approx. 7% of the fat was lost. The selective loss of fat and the minimal impact on phospholipids is a benefit in most applications of the whey-derived composition of the invention.

The inventors have also found that the content of phospholipids relative to total solids typically increases in the whey-derived product stream due to the germ-filtration, which fits well with the results of Table 2 which indicate a higher tendency to fat removal than to phospholipid removal.

The inventors have additionally observed that the content of polyunsaturated fatty acids relative to total fatty acids also tend to increase when the whey-derived product stream is germ-filtered.

### Reduction in Colony-Forming Units Due to Microfiltration

Nine batches of WPC70 feed were processed a) in a large-scale implementation of Example 1 but without the germ-filtration (“HT only”), and b) in the same large-scale implementation of Example 1 including both the MF-based germ-filtration followed by the heat-treatment (“MF+HT”). The obtained whey-derived powders were subsequently analyzed with respect to their content of colony forming units (CFU) per gram solids and the results are reported in Table 3. The large-scale implementation of Example 1 used the same parameter settings as Example 1 incl. membrane pore size and heating temperatures but was adapted to processing of larger quantities WPC70-feed.

TABLE 3
Impact of MF-based germ filtration on 9 batches of WPC feed
processed in a large-scale implementation of Example 1.

| | Heat only | MF + Heat |
|---|---|---|
| Batch | (CFU/g solids) | (CFU/g solids) |
| 1 | 2100 | <100 |
| 2 | 2800 | <100 |
| 3 | 4500 | <100 |
| 4 | 3400 | <100 |
| 5 | 3100 | <100 |
| 6 | 4300 | <100 |
| 7 | 4300 | <100 |
| 8 | 2600 | <100 |
| 9 | 2500 | <100 |

CFU = colony forming units. CFU contents of “<100” could even be zero.

When the combination of MF-based germ filtration and heat-treatment was applied, the CFU content of the whey-derived powders were consistently less than 100 CFU/g solids. Additionally, the contents of Bacillus cereus (incl. spores) were consistently less than 10 CFU/g solids.

In addition to the significant reduction in CFUs the inventors also observed that the content of endotoxin surprisingly was reduced significantly due to the germ filtration. The combination of MF-based germ filtration and heat-treatment therefore made it possible to produce phospholipid enriched, whey-derived products having a very low concentration of endotoxin and even phospholipid enriched, whey-derived products that are virtually free of endotoxin. This is surprising as endotoxins have a molecular size that would not be expected to be retained by an MF-based germ filtration. The inventors speculate that this effect is caused by the removal of microorganisms by the MF-based germ filtration, which microorganisms might have released enterotoxins if allowed to stay in the whey-derived product.

### Conclusion

The results described in the present Example document that the process streams for producing phospholipid enriched, whey-derived products advantageously can be subjected to a combination of MF-based germ filtration and gentle heat-treatment. This allows for an efficient reduction in the microbial content of the phospholipid enriched, whey-derived products while keeping the high nutritional quality of product.

### Example 4—Discovery and Investigation of Milk-Derived Extracellular Vesicles in Products of Example 1

Electron microscope-based investigations of the process streams and final WDP (in reconstituted form) of Example 1 revealed the presence of a significant amount of milk extracellular vesicles (milk EV) which surprisingly had survived the whey processing steps and also both the MF-based microbial reduction and the final heat-treatment.

In order to investigate the prevalence and abundance of EVs relative to milk fat globule membrane-material in the product of the invention, the novel WDP of Example 1 was analysed according to Analysis 2 to estimate the percentage of phospholipid source derived from EVs in the WDP.

### Procedure

The novel WDP of Example 1 was analysed according to Analysis 2 which is designed to determine the percentage of EV-derived phospholipids (PL) relative to total PL.

### Results and Discussion

The WDP of Example 1 was found to contain approx. 59% EV-derived PL relative to total PL, which is in line with the above-mentioned electron microscope observations of EVs in the product streams and final powder of Example 1. Comparable percentages of EV-derived PL found in isolated milk EVs, skimmed milk powder, cream, and sweet buttermilk powder (BMP) are also shown in Table 4. As would be expected, the phospholipid of BMP and cream consists primarily of phospholipid derived from the milk fat globule membrane and therefore have relatively small contribution from milk EV. In the opposite end of the spectrum, both skimmed milk powder and the EV isolate prepared from fresh milk had a very high phospholipid contribution from EVs. The inventors have also noted that the weight ratio between MFGM-derived PL and EV-derived PL of the WDP of Example 1 was approximately 1:1. The underlying BTN/CD9-ratio of the WP powder (i.e. the BTN/CD9-ratio used for calculating the EV-contribution to the total phospholipid content; see Analysis 2 for more details) corresponds very well to the BTN/CD9 ratio found in human milk (see e.g. Sari et al; Comparative Proteomics of Human Milk From Eight Cities in China During Six Months of Lactation in the Chinese Human Milk Project Study; Front. Nutr., 12 Aug. 2021 Sec.Food Chemistry https://doi.org/10.3389/fnut.2021.682429), indicating that the phospholipid of the new WDP has a composition, and an origin, very similar to the human milk phospholipid pool.

TABLE 4
Percentage of milk EV-derived phospholipids relative
to total phospholipids in different dairy products.
The percentages are determined according to Analysis 2.

| | % EV-derived PL |
|---|---|
| | relative to |
| Dairy stream | total PL (%) |
| Pure EVs isolated from fresh bovine milk | 100% |
| Skimmed milk powder | 80% |
| The novel WDP of Example 1 (according to | 59% |
| the invention) | |
| Cream | 24% |
| Sweet buttermilk powder | 5% |
| Pure MFGM isolated from fresh bovine milk | Approx. 0% |

### Conclusion

The inventors have seen (by electron microscopy imaging) a significant amount of apparently intact extracellular vesicles in the process streams and reconstituted powder product of Example 1. Using integral membrane proteins as markers for extracellular vesicles and milk fat globule membrane material, the inventors have determined that approx. 59% of the PL of the WDP of Example 1 was derived from extracellular vesicles. The phospholipid composition of the present product appears to be very similar to that of human milk and therefore well-suited for e.g. infant nutrition.

### Example 5—Discovery of the Presence of Human Milk microRNA in Products of Example 1

The inventors investigated to which extent the microRNA (miRNA) load of the milk EVs was still present in the whey-derived (WD) product streams of Example 1. miRNAs are small ˜22 nucleotide RNA sequences that can regulate target gene expression through sequence complementarity, binding and subsequent mRNA transcript degradation. Milk extracellular vesicles are an abundant source of miRNAs and scientific literature points to milk miRNAs being central regulators of infant gastrointestinal health and immune system development (Leroux et al). Human EV derived miRNAs survive gastrointestinal passage for recipient cell uptake in the gastrointestinal tract (Liao et al.). On the other hand, conventional infant formulas of today are largely devoid of bovine milk miRNAs (Leiferman et al) due to the conventional harsh processing steps leading to EV disintegration and release of miRNAs into the surrounding liquid whey space that contains endogenous RNAses that quickly degrade the free miRNAs. The containment of miRNAs within EVs represents therefore a protective environment against RNase degradation of miRNAs and ensures their perseverance. The quantification of miRNAs before and after specific processing steps can therefore be used as a surrogate marker for EV integrity and bioactive potential in a final infant formula.

### Procedure

The following four samples were analysed in this study:
Whey-derived liquid (WDL): the germ filtered, heat-treated MF permeate of Example 1 (3 replicates)
WDP: the whey-derived powder of Example 1 (3 replicates)
EV: laboratory-purified extracellular vesicles (EVs) isolated from skim milk using size-exclusion chromatography (according to doi.org/10.1080/20013078.2017.1294340) (3 replicates)
MFG: milk fat globules (MFG) isolated from the cream phase of bovine milk according to Hvarregaard et al) (3 replicates)

RNA was isolated from the four samples using RNeasy kit (Qiagen). RNA concentrations were measured and the isolated RNA was prepared for small RNA sequencing using Qiagen's QIAseq small RNA Library Prep kit. The finished libraries were quality controlled using an Agilent Bioanalyzer 2100 and quantified by use of qPCR. The three technical replicates of libraries were pooled and sequenced on an Illumina NextSeq500 sequencer.

The raw data from sequencing was quality filtered and trimmed using the fastxtoolkit and adaptors removed using cutadapt. Quality control was performed using FastQC to ensure high quality scores and expected length profiles. Filtered sequencing reads were mapped to a successive list of relevant transcriptomics datasets in order to identify small RNAs of relevance. The order of mapping involved:
1. All reads were mapped on bovine tRNA
2. The reads that had no match with bovine tRNA were then mapped on bovine micro RNA (miRNA)
3. The reads that had no match with tRNA and miRNA, was mapped against bovine small nuclear RNAs (snRNA)
4. Leftover reads were mapped into bovine ribosomal RNA (rRNA)
5. Leftover reads were mapped to bovine messenger RNA (mRNA)

### Results

The number of unique miRNAs were identified in the samples. The term “unique miRNAs” is used to specify how many different annotated miRNAs are seen, irrespective of expression level. Moreover, the relative abundance was assessed by number of sequencing reads. In general, the number of unique miRNAs was most stable in replicates of the MFG samples and EV samples, while more fluctuations were seen in the WDP/WDL replicate samples (liquid and powder). Nevertheless, >180 unique miRNAs were detected in both the WDL and WDP.

The miRNAs were next ranked in each sample type (calculated from the average of 3 replicate samples) according to abundance based on number of sequencing reads. The results are shown in Table 5. The identified miRNA species were compared to the 20 most abundant miRNA species in extracellular vesicles derived from human milk according to Herwijnen et al. Comparing the top 20 abundant human EV-miRNAs and top 20 abundant bovine EV-miRNAs in the tested samples, revealed several overlapping miRNAs that are 1) identical (human vs bovine), 2) Among the most abundant miRNAs, and 3) with preserved integrity after the processing steps as described in Example 1 (underlined miRNAs in Table 5).

As miRNAs are sensitive to both processing conditions and endogenous RNAses in the whey, it was surprising to find that among 291 unique miRNAs identified in gently purified (lab-scale) bovine EVs, 204 of these unique miRNAs were detected in the WDL (70%) and 191 of these unique miRNAs were detected in the WDP (66%). Moreover, assessing the identity of the top 20 abundant miRNAs present in human milk EVs (Herwijnen at al.), it was found that with regards to bovine milk EVs (gently lab-scale purified) and WDL and WDP, a large overlap of identical miRNAs were identified in both the WDL and WDP (Table 5, underlined miRNAs).

TABLE 5
The most abundant miRNAs that were detected in the analysed samples
(mean of replicates). miRNAs that are also found among the highest
ranking miRNAs of human milk have been underlined.

| Rank | MFG ref_{(lab-scale)} | EV ref_{(lab-scale)} | WDL | WDP |
|---|---|---|---|---|
| 1 | miR-191 | let-7a-5p | let-7f | let-7f |
| 2 | let-7b | let-7b | let-7a-5p | let-7a-5p |
| 3 | miR-223 | let-7f | let-7b | miR-223 |
| 4 | miR-148a | miR-21-5p | let-7i | let-7i |
| 5 | miR-200c | miR-148a | miR-223 | let-7b |
| 6 | miR-125b | let-7i | miR-21-5p | miR-21-5p |
| 7 | miR-151-3p | miR-3613a | miR-486 | miR-486 |
| 8 | miR-21-5p | let-7c | miR-199a-3p | miR-103 |
| 9 | miR-30d | miR-200c | miR-26b | miR-26b |
| 10 | miR-29c | miR-223 | miR-103 | miR-199a-3p |
| 11 | miR-30a-5p | miR-486 | miR-26a | miR-16b |
| 12 | miR-25 | miR-26a | miR-423-3p | miR-26a |
| 13 | let-7a-5p | miR-141 | miR-16b | miR-191 |

WDL = the whey-derived liquid product of Example 1; WDP = the whey-derived powder product of Example 1.

It was furthermore observed that the miRNA expression levels were very consistent internally in each sample even though read distributions showed variable miRNA percentages. This means that the variable miRNA percentages does not seem to affect the miRNA profiles. WDL and WDP and the EV sample had similar profiles, while the miRNA profile observed in relation to the MFG sample was more distant.

### Conclusion

It was found that both the WDP and the WDL of the invention contained significant amounts of miRNAs (65-70% of a gently lab-scale isolation method) of which 5-6 of the 20 most abundant miRNA species are identical to miRNAs from human milk EVs. Milk-derived miRNAs are known to play important roles in relation to infant development (see e.g. Leroux et al) and the present invention therefore enables the preparation of paediatric nutrition, and particularly the preparation of infant nutrition, enriched with respect to miRNA species found in human milk. As conventional infant formulas of today are largely devoid of miRNAs, this represents a ‘humanization gap’. The fact that the present inventors have documented preserved EV structures and miRNAs in both the WDL and WDL, emphasizes the suitability of employing the WDL or WDP for further humanization of infant nutrition in terms of bioactive EVs with intact miRNA content.

### SELECTED REFERENCES

Benmoussa A, Ly S, Shan S T, Laugier J, Boilard E, Gilbert C, Provost P: A subset of extracellular vesicles carries the bulk of microRNAs in commercial dairy cow's milk. J Extracell Vesicles. 2017 Nov. 21; 6(1):1401897.
Benmoussa A, Provost P: Milk MicroRNAs in Health and Disease. Compr Rev Food Sci Food Saf. 2019 May; 18(3):703-722.
Garcia-Martinez J, et al; Beneficial Effects of Bovine Milk Exosomes in Metabolic Interorgan Cross-Talk; Nutrients. 2022 Mar. 30; 14(7):1442.
Golan-Gerstl R, Shiff Y E, Moshayoff V, Schecter D, Leshkowitz D, Reif D:Characterization and biological function of milk-derived miRNAs. Mol Nutr Food Res. 2017 October; 61(10).
Herwijnen et al; 2018; Abundantly Present miRNAs in Milk-Derived Extracellular Vesicles Are Conserved Between Mammals. Front. Nutr. 5:81. doi: 10.3389/fnut.2018.00081
Hvarregaard et al; European Journal of Biochemistry CHARACTERIZATION OF GLYCOPROTEIN PAS-6/7 FROM MEMBRANES OF BOVINE MILK FAT GLOBULES; September 1996; https://doi.org/10.1111/1.1432-1033.1996.0628h.x
Leiferman A, Shu J, Upadhyaya B, Cui J, Zempleni J: Storage of Extracellular Vesicles in Human Milk, and MicroRNA Profiles in Human Milk Exosomes and Infant Formulas. J Pediatr Gastroenterol Nutr. 2019 August; 69(2):235-238.
Leroux C, Chervet M L, German J B: Perspective: Milk microRNAs as Important Players in Infant Physi-ology and Development. Adv Nutr. 2021 Oct. 1; 12(5):1625-1635.
Liao Y, Du X, Li J, Lönnerdal B: Human milk exosomes and their microRNAs survive digestion in vitro and are taken up by human intestinal cells. Mol Nutr Food Res. 2017 November; 61(11).
Lâsser C, Alikhani V S, Ekstrom K, Eldh M, Paredes P T, Bossios A, Sjöstrand M, Gabrielsson S, Lötvall J, Valadi H: Human saliva, plasma and breast milk exosomes contain RNA: uptake by macrophages. J Transl Med. 2011 Jan. 14; 9:9.
Smyczynska U, Bartlomiejczyk M A, Stanczak M M, Sztromwasser P, Wesolowska A, Barbarska 0, Pawlikowska E, Fendler W: Impact of processing method on donated human breast milk microRNA content. PLoS One. 2020 Jul. 15; 15(7):e0236126.
Zonneveld M I, van Herwijnen M J C, Fernandez-Gutierrez M M, Giovanazzi A, de Groot A M, Kleinjan M, van Capel T M M, Sijts A J A M, Taams L S, Garssen J et al: Human milk extracellular vesicles target nodes in interconnected signalling pathways that enhance oral epithelial barrier function and dampen immune responses. J Extracell Vesicles. 2021 March; 10(5):e12071.

1. A method of preparing a whey-derived composition enriched with respect to phospholipid, and preferably also enriched with respect to other milk fat globule membrane components, the method comprising the steps of:
a) providing a liquid feed comprising whey protein including alpha-lactalbumin (ALA) and phospholipid originating from whey, the liquid feed preferably having a pH in the range of 4.0-8, more preferably 5.5-7.5, even more preferably 5.7-7.0, and most preferably 5.9-6.6.
b) subjecting the liquid feed to membrane filtration to provide a filtration retentate and a filtration permeate, said membrane filtration is arranged and operated to:
provide a content of total phospholipid relative to total solids of the filtration retentate that is at least 200% of the content of total phospholipid relative to total solids of the liquid feed, and
provide a content of alpha-lactalbumin on total protein basis of the filtration retentate that is at most 75% of the content of alpha-lactalbumin on total protein basis of the liquid feed, and
c) subjecting the filtration retentate or a product stream comprising at least lipid and protein originating from the filtration retentate to microfiltration using a MF membrane with a pore size in the range of 1.0-2 micrometer and recovering the permeate of the microfiltration, which permeate may be subjected to further processing, and said further processing preferably comprising one or more additional processing steps, preferably comprising one or more of:
ii) concentration,
iii) heat-treatment, and
iv) drying.

2. The method according to any of the preceding claims wherein the whey of step a) is a sweet whey or an acid whey.

3. The method according to any of the preceding claims wherein the liquid feed comprises total protein in an amount in the range of 5-89% w/w relative to total solids, more preferably 30-86% w/w, even more preferably 40-83% w/w, and most preferably 60-80% w/w relative to total solids.

4. The method according to any of the preceding claims wherein the liquid feed comprises a total amount of alpha-lactalbumin in the range of 5-40% w/w relative to total protein, more preferably 10-35% w/w, even more preferably 10-30% w/w, and most preferably 10-25% w/w relative to total protein.

5. The method according to any of the preceding claims wherein the liquid feed comprises a total amount of phospholipid in the range of 0.2-5% w/w relative to total solids, more preferably 0.4-4% w/w, even more preferably 0.5-3% w/w, and most preferably 1-3% w/w relative to total solids.

6. The method according to any of the preceding claims wherein the liquid feed comprises a total amount of phospholipid derived from milk extracellular vesicles (milk EV) in an amount of at least 50% w/w relative to total phospholipid, more preferably at least 54% w/w, even more preferably at least 56% w/w, and most preferably at least 58% w/w.

7. The method according to any of the preceding claims wherein the liquid feed comprises a total amount phospholipid derived from milk EV in an amount of 50-75% w/w relative to total phospholipid, more preferably 54-73% w/w, even more preferably 56-71% w/w, and most preferably 58-70% w/w.

8. The method according to any of the preceding claims wherein the liquid feed comprises a total amount phospholipid derived from milk EV in an amount of at least 76% w/w relative to total phospholipid, more preferably at least 80% w/w, even more preferably at least 85% w/w, and most preferably at least 90% w/w.

9. The method according to any of the preceding claims wherein the liquid feed comprises total protein in an amount of 0.2-8% w/w relative to the weight of the liquid feed, more preferably 1-7% w/w, even more preferably 2-6% w/w, and most preferably 2-5% w/w relative to the weight of the liquid feed.

10. The method according to any of the preceding claims wherein the membrane filtration of step b) is arranged and operated to provide a content of alpha-lactalbumin on total protein basis of the filtration retentate that is at most 50% of the content of alpha-lactalbumin on total protein basis of the liquid feed, more preferably at most 30%, even more preferred at most 20% and most preferred at most 10% of the content of alpha-lactalbumin on total protein basis of the liquid feed.

11. The method according to any of the preceding claims wherein the membrane filtration of step b) is arranged and operated to provide a content of total phospholipid relative to total solids of the filtration retentate that is at least 225%, even more preferably at least 250%, and most preferably at least 275% of the content of total phospholipid relative to total solids of the liquid feed.

12. The method according to any of the preceding claims wherein the membrane filtration of step b) is arranged and operated to provide a content of osteopontin on total protein basis of the filtration retentate that is at least 180% of the content of osteopontin on total protein basis of the liquid feed, more preferably at least 200%, even more preferably at least 230%, and most preferably at least 250% of the content of osteopontin on total protein basis of the liquid feed.

13. The method according to any of the preceding claims wherein the membrane filtration of step b) involves one or more membrane(s) with a nominal molecular weight cut-off in the range of 100-2000 kDa, more preferably 300-1600 kDa; even more preferably 500-1300 kDa, and most preferably 700-1000 kDa.

14. The method according to any of the preceding claims wherein the membrane filtration of step b) involves diafiltration.

15. The method according to any of the preceding claims wherein the membrane filtration of step b) is operated with
with a trans-membrane pressure of 0.1-5 bar, more preferably 0.2-3 bar and most preferably 0.3-1 bar, and/or
at a temperature of 1-60 degrees C., more preferably 2-30 degrees C., even more preferably 5-20 degrees C., and most preferably 8-15 degrees C.

16. The method according to any of the preceding claims wherein the microfiltration of step c) uses an MF membrane with a pore size in the range of 1.2-1.8 micrometer.

17. The method according to any of the preceding claims wherein the microfiltration of step c) is performed with diafiltration.

18. The method according to claim 17 wherein the diafiltration uses water as diluent.

19. The method according to claim 30a wherein the microfiltration of step c) is operated with a trans-membrane pressure of 0.1-10 bar, more preferably 0.2-5 bar and most preferably 0.3-1 bar.

20. The method according to claim 30a wherein the microfiltration of step c) is operated at a temperature of 1-60 degrees C., more preferably 2-30 degrees C., even more preferably 5-20 degrees C., and most preferably 8-15 degrees C.

21. The method according to any of the preceding claims wherein the recovered permeate of the microfiltration of step c) is subjected to further processing, preferably comprising one or more of:
ii) concentration,
iii) heat-treatment, and
iv) drying.

22. The method according to any of the preceding claims wherein the further processing of step c) comprises:
ii) concentration, preferably using ultrafiltration, nanofiltration and/or reverse osmosis

23. The method according to any of the preceding claims wherein the further processing of step c) comprises:
iii) heat-treatment.

24. The method according to any of the preceding claims wherein the heat-treatment of step c-iii) involves heating the liquid to be heat-treated to a temperature of at least 60 degrees C. for a duration sufficient for obtaining at least partial microbial reduction, but wherein the heat-treatment denatures at most 5% of the BLG of the liquid to be heat-treated.

25. The method according to any of the preceding claims wherein the heat-treatment of step c-iii) involves heating the liquid to be heat-treated to a temperature of at least 70 degrees C. for a duration sufficient for obtaining at least partial microbial reduction, but wherein the heat-treatment denatures at most 20% of the BLG of the liquid to be heat-treated, more preferably at most 10% of the BLG, even more preferably at most 5% of the BLG and most preferably at most 1% of the BLG.

26. The method according to any of the preceding claims wherein the further processing of step c) comprises:
iv) drying, preferably comprising spray-drying.

27. The method according to any of the preceding claims wherein the further processing of step c) comprises:
ii) concentration, preferably using ultrafiltration, nanofiltration and/or reverse osmosis, followed by
iii) heat-treatment, preferably involving heating the liquid to be heat-treated to a temperature of at least 60 degrees C. for a duration sufficient for obtaining at least partial microbial reduction, but wherein the heat-treatment denatures at most 5% of the BLG of the liquid to be heat-treated, and followed by
iv) drying, preferably comprising or even consisting of spray-drying.

28. A whey-derived composition comprising:
total lipid in the amount of 10 to 30% w/w relative to total solids,
total phospholipid in an amount of 3 to 12% w/w relative to total solids,
an ash content in the range of 1-10% w/w relative to total solids,
lactose in the amount of at most 10% w/w relative to total solids,
total protein in an amount of 65 to 80% w/w relative to total solids,
a content of colony forming units (CFU) of at most 10000 CFU/g total solids.

29. The whey-derived composition according to claim 28 comprising at most 6000 CFU/g total solids, even more preferably at most 3000 CFU/g total solids, and most preferably the whey-derived composition contains at most 1000 CFU/g total solids.

30. The whey-derived composition according to claim 28 or 29 comprising a total amount of phospholipid derived from milk extracellular vesicles (milk EV) in an amount of at least 50% w/w relative to total phospholipid, more preferably at least 54% w/w, even more preferably at least 56% w/w, and most preferably at least 58% w/w relative to total phospholipid.

31. The whey-derived composition according to any of the claims 28-30 comprises a total amount phospholipid derived from milk EV in an amount of 50-75% w/w relative to total phospholipid, more preferably 54-73% w/w, even more preferably 56-71% w/w, and most preferably 58-70% w/w.

32. The whey-derived composition according to any of the claims 28-30 comprises a total amount phospholipid derived from milk EV in an amount of at least 76% w/w relative to total phospholipid, more preferably at least 80% w/w, even more preferably at least 85% w/w, and most preferably at least 90% w/w.

33. The whey-derived composition according to any of the claims 28-32 comprising milk EV, and preferably intact milk EV.

34. The whey-derived composition according to any of the claims 28-33 comprising microRNA (miRNA), preferably miRNA present in mammal milk, and most preferably miRNA present in bovine milk and/or in human milk.

35. The whey-derived composition according to claim 34 wherein the miRNA comprises a plurality of miRNA species, said plurality of miRNA species comprising at least one miRNA species selected from the group consisting of let-7a-5p, let-7b, let-7f, let-7i, miR-103, miR-16b, miR191, miR-199a-3p, miR-21-5p, miR-223, miR-26a, miR-26b, miR-423-3p, and miR-486.

36. The whey-derived composition according to claim 34 wherein the miRNA comprises a plurality of miRNA species, said plurality of miRNA species comprising let-7a-5p, let-7b, let-7f, let-7i, miR-103, miR-16b, miR-191, miR-199a-3p, miR-21-5p, miR-223, miR-26a, miR-26b, miR-423-3p, and miR-486.

37. The whey-derived composition according to claim 34 wherein the miRNA comprises a plurality of miRNA species, said plurality of miRNA species comprising at least one miRNA species selected from the group consisting of let-7a-5p, let-7b, let-7f, miR-191, miR-21-5p, and miR-26a.

38. The whey-derived composition according to claim 34 wherein the miRNA comprises a plurality of miRNA species, said plurality of miRNA species comprising the miRNA species let-7a-5p, let-7b, let-7f, miR-191, miR-21-5p, and miR-26a.

39. The whey-derived composition according to any of the claims 28-38 in the form of a powder.

40. The whey-derived composition according to any of the claims 28-38 in the form of a liquid.

41. The whey-derived composition according to any of the claims 28-40 obtainable by a method according to one or more of claims 1-27.

42. Use of the whey-derived composition according to any of the claims 28-41 as a food ingredient, preferably for increasing the content of OPN in a nutritional product, and preferably wherein the nutritional product is a paediatric product and more preferably an infant formula; preferably using the whey-derived composition in an amount sufficient to provide a content of OPN of to the nutritional product of at least 10 mg/100 g total solids of the nutritional product, more preferably at least 20 mg/100 g total solids, even more preferably at least 30 mg/100 g total solids, and most preferably at least 40 mg/100 g total solids.

43. Use of the whey-derived composition according to any of the claims 28-41 as a food ingredient, preferably for increasing the content of extracellular vesicles in a nutritional product, and preferably wherein the nutritional product is a paediatric product and more preferably an infant formula.

44. Use of the whey-derived composition according to any of the claims 28-41 as a food ingredient, for increasing the content of miRNA, preferably miRNA present in mammal milk, and most preferably miRNA present in bovine milk and/or in human milk, in a nutritional product, and preferably wherein the nutritional product is a paediatric product and more preferably an infant formula.

45. The use according to claim 44 wherein the miRNA comprises a plurality of miRNA species, said plurality of miRNA species comprising at least one miRNA species selected from the group consisting of let-7a-5p, let-7b, let-7f, let-7i, miR-103, miR-16b, miR-191, miR-199a-3p, miR21-5p, miR-223, miR-26a, miR-26b, miR-423-3p, and miR-486.

46. The use according to claim 44 wherein the miRNA comprises a plurality of miRNA species, said plurality of miRNA species comprising let-7a-5p, let-7b, let-7f, let-7i, miR-103, miR-16b, miR-191, miR-199a-3p, miR-21-5p, miR-223, miR-26a, miR-26b, miR-423-3p, and miR-486.

47. The use according to claim 44 wherein the miRNA comprises a plurality of miRNA species, said plurality of miRNA species comprising at least one miRNA species selected from the group consisting of let-7a-5p, let-7b, let-7f, miR-191, miR-21-5p, and miR-26a.

48. The use according to claim 44 wherein the miRNA comprises a plurality of miRNA species, said plurality of miRNA species comprising the miRNA species let-7a-5p, let-7b, let-7f, miR-191, miR-21-5p, and miR-26a.

49. The use according to any of claims 42-48, wherein the whey-derived composition is used in an amount sufficient to provide a content of solids to the nutritional product of at least 0.1 g/100 g total solids of the nutritional product, more preferably at least 0.5 g/100 g total solids, even more preferably at least 2 g/100 g total solids, and most preferably at least 3 g/100 g total solids.

50. The use according to any of claims 42-49, wherein the whey-derived composition is used in an amount sufficient to provide a content of solids to the nutritional product of 0.1-30 g/100 g total solids of the nutritional product, more preferably 0.5-20 g/100 g total solids, even more preferably 2-15 g/100 g total solids, and most preferably 3-12 g/100 g total solids.

51. A nutritional product, which preferably is a paediatric product, and more preferably an infant formula, comprising the whey-derived composition according to one or more of claims 28-41 in an amount sufficient to:
to provide a content of solids to the nutritional product of 0.1-30 g/100 g total solids of the nutritional product, more preferably 0.5-20 g/100 g total solids, even more preferably 2-15 g/100 g total solids, and most preferably 3-12 g/100 g total solids.
